(19) United States
(12) Patent Application Publication
(10) Pub. No.: US 2014/0228435 A1
(43) Pub. Date: Aug. 14, 2014

(54) COMPOSITION AND METHOD OF SYNTHESIZING A BIOMOLECULE AND ITS THERAPEUTICS APPLICATIONS

(71) Applicant: MEHDI BAKHTIARI, KARAJ (IR)

(72) Inventor: MEHDI BAKHTIARI, KARAJ (IR)

(21) Appl. No.: 13/765,738

(22) Filed: Feb. 13, 2013

(51) Int. Cl. A61K 31/197; C07C 229/26

(52) U.S. Cl. CPC A61K 31/197; C07C 229/26
USPC 514/556; 562/560

(57) ABSTRACT

The embodiments herein provide a therapeutic composition comprising N (delta)-protonated L-2-amino-5-diaminomethyleneamino-pentanoic acid called J Factor and a method of ex-vivo synthesis of J-Factor from N(omega)′-protonated L-arginine at a pH of 5-9. The method involves increasing a temperature and or decreasing an acidity of an aqueous solution containing the N(omega)′-protonated L-arginine to obtain a pH value of greater than pKa−2 or less than pKa+2. The derivatives of aqueous solution are allowed to reach an equilibrium state at 25° C. The acidity of the acidity of the aqueous solution is adjusted to a pH value of 7.0 at 25° C. The pKa is a minus logarithm of an acid dissociation constant of the N(omega)′-protonated guanidino group. The N(omega)′-protonated 1-arginine includes ionized forms in the carboxyl or 2-amino group, which are in equilibrium with the N(omega)′-protonated 1-arginine. The composition has a molecular mass of more than or equal to 175.2u

### CROSS REFERENCE TO RELATED APPLICATIONS

This patent application claims priority to U.S. Non-Provisional patent application Ser. No. 12/690,126, filed Jan. 20, 2010, which is incorporated by reference in its entirety.

### BACKGROUND

1. Technical Field

The embodiments herein generally relates to the field of physiology. The embodiments particularly relates to an ex-vivo synthesis of a bio molecule found in-vivo and involved in the signaling and stimulation of autocrine-paracrine Insulin like Growth Factor-1 (IGF-I) secretion in different tissues in the body. The embodiments more particularly relates to an ex-vivo synthesis of a biomolecule called J-Factor and its medical application.

2. Description of the Related Art

Most aging individuals die of various health related issues. During old age, a loss of muscle strength and a weakening of bone results in frailty, thereby limiting an individual's chances of living an independent life until death. The physical changes during aging have been considered physiologic but there is evidence that some of these changes are related to a decline in the hormonal activity. Science recognizes aging as a disease that can be reversed to a large degree by increasing the Growth Hormone (GH) levels. The biological aging is closely associated with a decline in the capacity for protein synthesis which has been hypothesized to contribute to the decline in a tissue function and increased susceptibility to disease. GH and Insulin-Like Growth Factor-1 (IGF1) are important anabolic hormones that regulate the metabolic processes such as a protein synthesis in almost all tissues throughout a life span. GH is also required for a normal postnatal growth, having a critical role in a bone growth as well as important regulatory effects on protein, carbohydrate, and lipid metabolism. The physiological effects of GH are brought about by a GH receptor.

It was noticed that any kind of exercises such as jogging, running, bicycling, walking or skating reduced the body fat mass and increased the lean muscle mass. During the exercise, blood glucose is utilized by the body cells leading to a decrease in plasma/blood glucose, which in turn induced the growth hormone (GH) production thereby stimulating autocrine-paracrine IGF-1 signaling leading to the decrease in fat tissue by lipolysis, and strengthening and development of muscles-myogenesis. These changes effectively occurred after a delay from the time when the exercise is started and continued. In addition, there is a decrease in these changes after a delay from the time when the exercise is stopped.

In other words, during the course of the exercise, the above changes were not a gradual process from the beginning. This is because of an accumulation of ‘the factor’ responsible for inducing the changes at first in the cell. Thus the accumulated factor stimulates a production of autocrine-paracine IGF-1.

The Growth hormone harbors the above factor which is responsible for the production of autocrine-paracine IGF-1. The Growth hormone is broken down into its respective amino acids within the cell after an interaction with its receptor, wherein the factor is formed from one of the amino acids of GH, which begins to accumulate in the cell, and then stimulating the synthesis and secretion of IGF-1.

It is noticed that in a premature neonates with very low birth weight (VLBW), when fed with dried milk containing casein, the premature neonates are infected with late metabolic acidosis. In this condition, in spite of feeding milk, a weight gain and an increase in a fatty tissue does not happen due to the increase in urea. This indicates that urea causes a decrease in the fatty tissue.

When we consider or assume that stimulating the secretion of autocrine-paracrine IGF-1 by urea, is a reason for the decrease in the fatty tissue, the ‘Factor’ stimulating the secretion of IGF-1 should have the following moiety in its chemical structure without resonance (mesomerism)

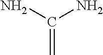

Due to the physiologic condition of the body (pH 7.40 and 37° C.), urea significantly has a chemical formula:

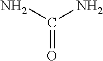

which is one of its tautomeric forms.

Due to the physiologic condition of the body (pH 7.40 and 37° C.), and with regard to a pKa of 0.18 at 25° C. in protonated urea with endothermic deprotonation, that undergoing resonance(mesomerism) isn't significantly observed under physiologic condition of the body (pH 7.40 and 37° C.). Thus, the protontated form of urea can't stimulate the synthesis and secretion of IGF-1. However, the length of the C—NH₂ bond equals 1.47 Å (Angstrom) in the chemical formula.

Since, the ‘Factor’ is one of the amino acids of GH, next step of the embodiments herein is to examine the amino acid of GH having the following moiety in its chemical structure without resonance (mesomerism).

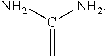

Moreover, when the length of the C—NH₂ bond didn't equal 1.47 Å (Angstrom) in the moiety, the ‘Factor’ wouldn't join its intracellular receptor to stimulate the synthesis and secretion of IGF-1. It's clear resonance make the length shorter than 1.47 Å.

It was found that none of the amino acids of extracellular GH before joining its receptor on the cell surface had the above shown moiety, but it was observed that when the double bond in the N(omega)′-protonated Guanidino group (NH₂—C(═NH₂⁺)—NH—) of L-arginine which is one of the amino acids of extracellular GH changes its place, then new bio-molecule having the following moiety in its chemical structure is possible. (Guanidino=diaminomethylideneamino)

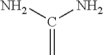

This new biomolecule having the above moiety in its structure is referred for this invention as N(delta)-protonated L-2-amino-5-diaminomethyleneamino-pentanoic acid or ‘J-Factor’.

Hence there is a need for an ex-vivo synthesis of a biomolecule with the above moiety called “J-Factor” or N(delta)-protonated L-2-amino-5-diaminomethyleneamino-pentanoic acid formulated at pH 5-9

The above mentioned shortcomings, disadvantages and problems are addressed herein and which will be understood by reading and studying the following specification.

### OBJECTIVES OF THE EMBODIMENTS

The primary object of the embodiments herein is to develop a method for an ex-vivo synthesis of a biomolecule called “J-Factor” or N(delta)-protonated L-2-amino-5-diaminomethyleneamino-pentanoic acid formulated at pH 5-9.

Another object of the embodiments herein is to sysnthesize a biomolecule called “J-Factor”or N (delta)-protonated L-2-amino-5-diaminomethyleneamino-pentanoic acid from N(omega)′-protonated L-arginine.

Yet another object of the embodiments herein is to develop a therapeutic composition comprising N (delta)-protonated L-2-amino-5-diaminomethyleneamino-pentanoic acid.

Yet another object of the embodiments herein is to develop a method of converting a composition having N(omega)′-protonated guanidino group to a product having N(delta)-protonated diaminomethyleneamino group.

Yet another object of the embodiments herein is to develop and a therapeutic composition comprising N(delta)-protonated L-2-amino-5-diaminomethyleneamino-pentanoic acid for improving a muscular hypertrophy, restoring a muscle mass, increasing bone density, decreasing fatty tissues, improving central vision of eyes, decreasing a cellular proptosis, improving skin elasticity, improving tone of skin and improving color of skin.

Yet another object of the embodiments herein is to develop and a therapeutic composition comprising N(delta)-protonated L-2-amino-5-diaminomethyleneamino-pentanoic acid which is administered orally or through injection and is synthesized easily.

Yet another object of the embodiments herein is to develop and a therapeutic composition comprising N(delta)-protonated L-2-amino-5-diaminomethyleneamino-pentanoic acid which is non-diabetogenic and non-antigenic.

Yet another object of the embodiments herein is to develop and a therapeutic composition comprising N(delta)-protonated L-2-amino-5-diaminomethyleneamino-pentanoic acid which exhibits no complication such as joint swelling, carpal tunnel syndrome and joint pain, when the composition is administered.

Yet another object of the embodiments herein is to develop and a therapeutic composition comprising N(delta)-protonated L-2-amino-5-diaminomethyleneamino-pentanoic acid in a suitable pharmaceutical which is acceptable for administering to patients suffering from age-related disorders and diseases.

Yet another object of the embodiments herein is to develop and a therapeutic composition comprising N(delta)-protonated L-2-amino-5-diaminomethyleneamino-pentanoic acid which is prepared for oral administration by mixing J-factor having the desired degree of purity with physiologically acceptable carriers that are non toxic to the recipients at the given dosages and concentrations.

Yet another object of the embodiments herein is to develop and a therapeutic composition comprising N(delta)-protonated L-2-amino-5-diaminomethyleneamino-pentanoic acid which is administered to patients selected from the group consisting of bone-fracture, wound healing, type-II diabetic, neurodegenerative conditions, cancer, aging, and muscle wasting diseases.

Yet another object of the embodiments herein is to develop and a therapeutic composition comprising N(delta)-protonated L-2-amino-5-diaminomethyleneamino-pentanoic acid which is mixed with L-agrinine having desired purity and suitable pharmaceutically acceptable carriers.

Yet another object of the embodiments herein is to develop and a therapeutic composition comprising N(delta)-protonated L-2-amino-5-diaminomethyleneamino-pentanoic acid which is beneficial and useful to reduce the signs of the aging and age-related disorders.

These and other objects and advantages of the embodiments herein will become readily apparent from the following detailed description taken in conjunction with the accompanying drawings.

### SUMMARY

The various embodiments herein provide a therapeutic composition comprising N (delta)-protonated L-2-amino-5-diaminomethyleneamino-pentanoic acid formulated at pH 5-9 and a method of ex-vivo synthesis of a biomolecule called “J-Factor” or N (delta)-protonated L-2-amino-5-diaminomethyleneamino-pentanoic acid from N(omega)′-protonated L-arginine. The embodiments herein provide an ex-vivo synthesis of a biomolecule found in-vivo and involved in the signaling and stimulation of the synthesis and secretion of autocrine-paracrine Insulin like Growth Factor-I (IGF-I) in different tissues in the body.

According to an embodiment herein, a therapeutic composition of N(delta)-protonated L-2-amino-5-diaminomethyleneamino-pentanoic acid formulated at pH 5-9 is provided. The therapeutic composition comprises N(delta)-protonated L-2-amino-5-diaminomethyleneamino-pentanoic acid having a structural formula represented by

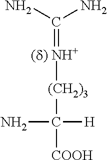

where N is Nitrogen, O is Oxygen, H is Hydrogen, C is Carbon, ═ is a double bond, — is a single bond, —NH2 represents an amino group, —CH2- represents Methylene, —COOH is a Carboxyl group, and wherein the therapeutic composition includes ionized forms of the N(delta)-protonated L-2-amino-5-diaminomethyleneamino-pentanoic acid in a carboxyl group or in a 2-amino group, and wherein the ionized forms are in equilibrium with the N(delta)-protonated L-2-amino-5-diaminomethyleneamino-pentanoic acid, and wherein a chemical structure of the therapeutic composition has a N(delta)-protonated L-2-amino-5-diaminomethyleneamino group, wherein the therapeutic composition has a second structural formula, and wherein the second structural formula is represented by

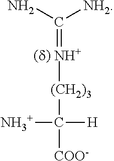

According to an embodiment herein, the therapeutic composition is derived from N(omega)′-protonated 1-arginine having a third structural formula and wherein the third structural formula is represented by

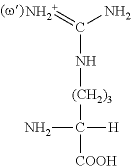

and wherein the therapeutic composition is derived from the N(omega)′-protonated 1-arginine by bringing a first Kelvin temperature of an aqueous solution of the N(omega)′-protonated 1-arginine to a second Kelvin temperature and increasing the acidity of the aqueous solution to obtain a preset pH value and wherein the second Kelvin temperature is less than ΔH°/(ΔS°+2R), and wherein the ΔH° is a change in an enthalpy and wherein the ΔS° is a change in an entropy under a standard temperature and pressure conditions and wherein the standard temperature and pressure conditions includes a temperature of 25° C. and a pressure of 1 atmospheric pressure, and wherein the R is gas constant and wherein the preset value of pH is less than a sum of pKa+2, and wherein the pKa is a minus logarithm of a first acid dissociation constant of a diprotonated guanidino group of an intermediate chemical compound having a fourth structural formula in the aqueous solution at the second Kelvin temperature and wherein the fourth structural formula is represented by

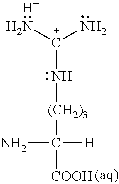

wherein the fourth structural formula includes its ionized forms in a carboxyl group or 2-amino group which are in equilibrium with the fourth structural formula, and then giving time to the derivatives of the N(omega)′-protonated 1-arginine to reach an equilibrium state at the pH which is less than the sum of pKa+2 at the second Kelvin temperature, and wherein said time doesn't need to be increased to more than 24 hours, and wherein the acidity of the aqueous solution is brought to a pH value of 7.0 and the temperature of the aqueous solution to 25° C. after reaching the equilibrium state, and wherein the N(omega)′-protonated 1-arginine includes its ionized forms in the carboxyl or 2-amino group which are in equilibrium with the N(omega)′-protonated 1-arginine, and wherein the N(omega)′-protonated 1-arginine has a N(omega)′-protonated guanidino group, and wherein the therapeutic composition stimulates a synthesis and secretion of autocrine-paracrine IGF-1 in tissues in a living body.

According to an embodiment herein, the therapeutic composition is produced by dislocating a double bond in the N(omega)′-protonated guanidino group of the N(omega)′-protonated L-arginine which is represented by a following reaction:

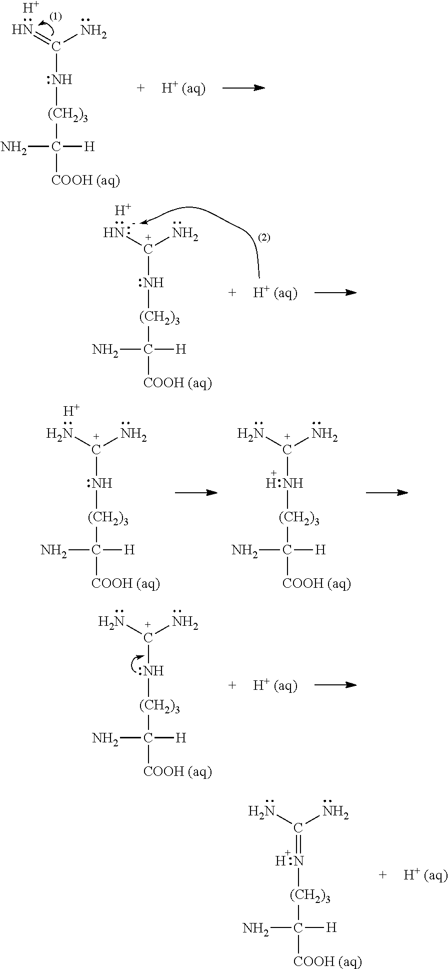

wherein the reaction is reversible at any stage.

According to an embodiment herein, the N(delta)-protonated diaminomethyleneamino group increases an expression of IGF-1mRNA in all cells of a living body.

According to an embodiment herein, the therapeutic composition is ex-vivo synthesized from N(omega)′-protonated L-arginine by increasing a temperature of an aqueous solution containing the N(omega)′-protonated L-arginine and/or decreasing an acidity of the aqueous solution containing the N(omega)′-protonated L-arginine to obtain a pH value which is greater than a value of pKa−2 and wherein the pKa is a minus logarithm of an acid dissociation constant of the N(omega)′-protonated guanidino group of the N(omega)′-protonated L-arginine in the aqueous solution at its temperature, allowing the derivatives of the N(omega)′-protonated L-arginine to reach an equilibrium state in the aqueous solution at the pH which is greater than the value of pKa−2 by providing time, and wherein said time doesn't need to be increased to more than 24 hours, and bringing the acidity of the aqueous solution to a pH value of 7.0 and the temperature of the aqueous solution to 25° C. after reaching the equilibrium state and wherein the N(omega)′-protonated 1-arginine includes its ionized forms in a carboxyl or 2-amino group, which are in equilibrium with the N(omega)′-protonated 1-arginine.

According to an embodiment herein, the therapeutic composition is ex-vivo synthesized from a reactant by bringing a temperature and an acidity of an aqueous solution with a pH which is greater than a value of pKa−2 sequentially to 25° C. and a pH value of 7.0, wherein the pKa is a minus logarithm of an acid dissociation constant of a N(omega)′-protonated guanidino group of N(omega)′-protonated L-arginine in the aqueous solution at its temperature, wherein the aqueous solution contains the reactant, and wherein the reactant is a sum of L-2-amino-5-diaminomethyleneamino-pentanoic acid and ionized forms of the L-2-amino-5-diaminomethyleneamino-pentanoic acid in a carboxyl group or 2-amino group which are in equilibrium with the L-2-amino-5-diaminomethyleneamino-pentanoic acid.

According to an embodiment herein, the therapeutic composition improves hair growth and muscular hypertrophy and restores the muscle mass, increases bone density, decreases fatty tissue, improves eye's central vision, decreases cellular proptosis, and improves skin elasticity

According to an embodiment herein, the therapeutic composition is synthesized from N(omega)′-protonated L-arginine through a process comprising the steps of dissolving the N(omega)′-protonated L-arginine in a liquid solvent system containing a strong acid with a known negative Hammett acidity function “H₀” to obtain a solution, bringing a first Kelvin temperature of the solution to a second Kelvin temperature, and wherein the second Kelvin temperature is less than ΔH°/(ΔS°−R(H₀−2)), and wherein the R is gas constant, and wherein the ΔH° is a change in enthalpy and wherein the ΔS° is a change in entropy change under a standard temperature and pressure conditions and wherein the standard temperature and pressure conditions includes a temperature of 25° C. and a pressure of 1 atmospheric pressure, when a N(omega)′-protonated guanidino group of the N(omega)′-protonated L-arginine is converted to a diprotonated form of the guanidino group in water, and wherein the maximum of the second Kelvin temperature is increased, when a value of the H₀ is reduced, allowing derivatives of the N(omega)′-protonated L-arginine to reach an equilibrium state in the solution at the second Kelvin temperature by providing time; and wherein said time doesn't need to be increased to more than 24 hours, and bringing the acidity of the solution to a pH value of 7.0 and the temperature of the solution to 25° C., wherein the N(omega)′-protonated 1-arginine includes its ionized forms in a carboxyl or 2-amino group, which are in equilibrium with the N(omega)′-protonated 1-arginine.

According to an embodiment herein, the therapeutic composition stimulates a synthesis and a secretion of IGF-1

According to an embodiment herein, a therapeutic compound formulated at pH 5-9 is provided. the therapeutic compound has a formula

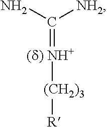

wherein the R′ is a chemical group bonding to the —(CH₂)₃— through a single C—C bond, and wherein the R′ contains a carboxyl group or an ionized form of the carboxyl group, and wherein the R′ contains an amino group or an ionized form of the amino group, and wherein the therapeutic compound has a molecular mass of more than or equal to 174.2 u.

According to an embodiment herein, the therapeutic compound is derived from a composition having a N(omega)′-protonated guanidino group through a method comprising the steps of dissolving the composition in a liquid solvent system containing a strong acid with a known negative Hammett acidity function “H₀” to obtain a solution, bringing a first Kelvin temperature of the solution to a second Kelvin temperature, and wherein the second Kelvin temperature is less than ΔH°/(ΔS°−R(H₀−2)), and wherein the R is gas constant, and wherein the ΔH° is a change in enthalpy and wherein the ΔS° is a change in entropy change under a standard temperature and pressure conditions and wherein the standard temperature and pressure conditions includes a temperature of 25° C. and a pressure of 1 atmospheric pressure, when the N(omega)′-protonated guanidino group is converted to a diprotonated form of the guanidino group in water, and wherein the maximum of the second Kelvin temperature is increased, when a value of the H₀ is reduced, allowing derivatives of the N(omega)′-protonated guanidino group to reach an equilibrium state in the solution at the second Kelvin temperature by providing time; and wherein said time doesn't need to be increased to more than 24 hours, and bringing the acidity of the solution to a pH value of 7.0 and the temperature of the solution to 25° C., wherein the composition has a structural formula

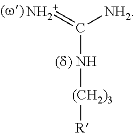

According to an embodiment herein, the therapeutic compound is derived from a composition having a N(omega)′-protonated guanidino group through a method comprising the steps of increasing a temperature of an aqueous solution containing the composition having the N(omega)′-protonated guanidino group and/or decreasing an acidity of the aqueous solution containing the composition having the N(omega)′-protonated guanidino group in structure to obtain a pH which is more than a value of pKa−2, and wherein the pKa is a minus logarithm of an acid dissociation constant of the N(omega)′-protonated guanidino group in the aqueous solution at its temperature, wherein the pKa−2 is reduced when the temperature is increased; giving time to derivatives of the N(omega)′-protonated guanidino group to reach an equilibrium state in the aqueous solution at the pH which is more than the value of pKa−2; and wherein said time doesn't need to be increased to more than 24 hours, and bringing the temperature of the aqueous solution to 25° C. and the acidity of the aqueous solution to a pH value of 7.0 after reaching the equilibrium state, and wherein the composition has a chemical structure represented by

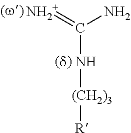

According to an embodiment herein, the therapeutic compound stimulates a synthesis and a secretion of IGF-1

According to an embodiment herein, a method of converting a composition having a N(omega)′-protonated guanidino group to a product having a N(delta)-protonated diaminomethyleneamino group is provided. The method comprises the steps of bringing a first Kelvin temperature of an aqueous solution containing the composition having the N(omega)′-protonated guanidino group to a second Kelvin temperature, and wherein the second Kelvin temperature is less than ΔH°/(ΔS°+2R), and wherein the ΔH° is a change in enthalpy and wherein the ΔS° is a change in entropy under a standard temperature and pressure conditions and wherein the standard temperature and pressure conditions include a temperature of 25° C. and a pressure of 1 atmospheric pressure, when the N(omega)′-protonated guanidino group is converted to a diprotonated form of the guanidine group in the aqueous solution, and wherein the R is gas constant; increasing an acidity of the aqueous solution containing the composition having the N(omega)′-protonated guanidino group to obtain a pH which is less than a value of pKa+2, and wherein the pKa is a minus logarithm of a first acid dissociation constant of the diprotonated form of the guanidino group of the composition at the second Kelvin temperature in the aqueous solution, and wherein the pKa+2 value is increased when the second Kelvin temperature is reduced; allowing derivatives of the N(omega)′-protonated guanidino group to reach an equilibrium state in the aqueous solution at the pH which is less than the value of pKa+2 together and at the second Kelvin temperature by providing time; and wherein said time doesn't need to be increased to more than 24 hours, and bringing the acidity of the aqueous solution to a pH value of 7.0 and the temperature of the aqueous solution to 25° C., wherein the composition has a formula represented by

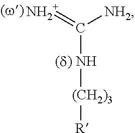

wherein the R′ is a chemical group bonding to the —(CH₂)₃— through a single C—C bond, and wherein the R′ contains a carboxyl group or an ionized form of the carboxyl group, and wherein the R′ contains an amino group or an ionized form of the amino group, and wherein the composition has a molecular mass of more than or equal to 174.2 u.

According to an embodiment herein, the N(delta)-protonated diaminomethyleneamino group stimulates a synthesis of IGF-1mRNA and IGF-1 in the cell.

According to an embodiment herein, the product is a therapeutic composition.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a N15 NMR spectra of crystalline L-arginine hydrochloride indicating an absence of resonance (mesomerism) between the C—N₁, C—N₃ and C—N (δ) bonds of N(omega)′-protonated L-arginine.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a therapeutic composition comprising N (delta)-protonated L-2-amino-5-diaminomethyleneamino-pentanoic acid and a method of ex-vivo synthesis of a biomolecule called “J-Factor” or N(delta)-protonated L-2-amino-5-diaminomethyleneamino-pentanoic acid from N(omega)′-protonated L-arginine. The embodiments herein provide an ex-vivo synthesis of a biomolecule found in-vivo and involved in the signaling and stimulation of the synthesis and secretion of autocrine-paracrine Insulin like Growth Factor-I (IGF-I) in different tissues in the body.

According to an embodiment herein, a therapeutic composition of N(delta)-protonated L-2-amino-5-diaminomethyleneamino-pentanoic acid formulated at pH 5-9 is provided. The therapeutic composition comprises N(delta)-protonated L-2-amino-5-diaminomethyleneamino-pentanoic acid having a structural formula represented by

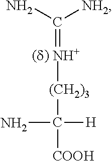

where N is Nitrogen, O is Oxygen, H is Hydrogen, C is Carbon, ═ is a double bond, — is a single bond, —NH2 represents an amino group, —CH2- represents Methylene, COOH is a Carboxyl group, and wherein the therapeutic composition includes ionized forms of the N(delta)-protonated L-2-amino-5-diaminomethyleneamino-pentanoic acid in a carboxyl group or in a 2-amino group, and wherein the ionized forms are in equilibrium with the N(delta)-protonated L-2-amino-5-diaminomethyleneamino-pentanoic acid, and wherein a chemical structure of the therapeutic composition has a N(delta)-protonated L-2-amino-5-diaminomethyleneamino group, wherein the therapeutic composition has a second structural formula, and wherein the second structural formula is represented by

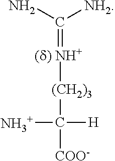

According to an embodiment herein, the therapeutic composition is derived from N(omega)′-protonated 1-arginine having a third structural formula and wherein the third structural formula is represented by

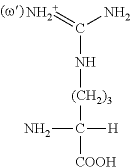

and wherein the therapeutic composition is derived from the N(omega)′-protonated 1-arginine by bringing a first Kelvin temperature of an aqueous solution of the N(omega)′-protonated 1-arginine to a second Kelvin temperature and increasing the acidity of the aqueous solution to obtain a preset pH value and wherein the second Kelvin temperature is less than ΔH°/(ΔS°+2R), and wherein the ΔH° is a change in an enthalpy and wherein the ΔS° is a change in an entropy under a standard temperature and pressure conditions and wherein the standard temperature and pressure conditions includes a temperature of 25° C. and a pressure of 1 atmospheric pressure, and wherein the R is gas constant and wherein the preset value of pH is less than a sum of pKa+2, and wherein the pKa is a minus logarithm of a first acid dissociation constant of a diprotonated guanidino group of an intermediate chemical compound having a fourth structural formula in the aqueous solution at the second Kelvin temperature and wherein the fourth structural formula is represented by

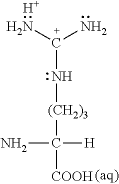

wherein the fourth structural formula includes its ionized forms in a carboxyl group or 2-amino group which are in equilibrium with the fourth structural formula, and then giving time to the derivatives of the N(omega)′-protonated 1-arginine to reach an equilibrium state at the pH which is less than the sum of pKa+2 at the second Kelvin temperature, and wherein said time doesn't need to be increased to more than 24 hours, and wherein the acidity of the aqueous solution is brought to a pH value of 7.0 and the temperature of the aqueous solution to 25° C. after reaching the equilibrium state, and wherein the N(omega)′-protonated 1-arginine includes its ionized forms in the carboxyl or 2-amino group which are in equilibrium with the N(omega)′-protonated 1-arginine, and wherein the N(omega)′-protonated 1-arginine has a N(omega)′-protonated guanidino group, and wherein the therapeutic composition stimulates a synthesis and secretion of autocrine-paracrine IGF-1 in tissues in a living body.

According to an embodiment herein, the therapeutic composition is produced by dislocating a double bond in the N(omega)′-protonated guanidino group of the N(omega)′-protonated L-arginine which is represented by a following reaction:

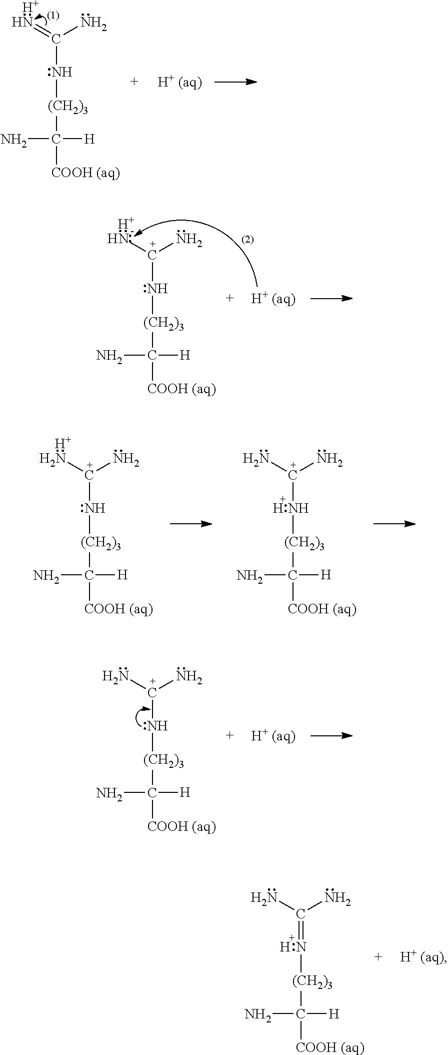

wherein the reaction is reversible at any stage.

According to an embodiment herein, the N(delta)-protonated diaminomethyleneamino group increases an expression of IGF-1mRNA in all cells of a living body.

According to an embodiment herein, the therapeutic composition is ex-vivo synthesized from N(omega)′-protonated L-arginine by increasing a temperature of an aqueous solution containing the N(omega)′-protonated L-arginine and/or decreasing an acidity of the aqueous solution containing the N(omega)′-protonated L-arginine to obtain a pH value which is greater than a value of pKa−2 and wherein the pKa is a minus logarithm of an acid dissociation constant of a N(omega)′-protonated guanidino group of the N(omega)′-protonated L-arginine in the aqueous solution at its temperature, allowing the derivatives of the N(omega)′-protonated L-arginine to reach an equilibrium state in the aqueous solution at the pH which is greater than the value of pKa−2 by providing time, and wherein said time doesn't need to be increased to more than 24 hours, and bringing the acidity of the aqueous solution to a pH value of 7.0 and the temperature of the aqueous solution to 25° C. after reaching the equilibrium state and wherein the N(omega)′-protonated 1-arginine includes its ionized forms in a carboxyl or 2-amino group, which are in equilibrium with the N(omega)′-protonated 1-arginine.

According to an embodiment herein, the therapeutic composition is ex-vivo synthesized from a reactant by bringing a temperature and an acidity of an aqueous solution with a pH which is greater than a value of pKa−2 sequentially to 25° C. and a pH value of 7.0, wherein the pKa is a minus logarithm of an acid dissociation constant of a N(omega)′-protonated guanidino group of N(omega)′-protonated L-arginine in the aqueous solution at its temperature, wherein the aqueous solution contains the reactant, and wherein the reactant is a sum of L-2-amino-5-diaminomethyleneamino-pentanoic acid and ionized forms of the L-2-amino-5-diaminomethyleneamino-pentanoic acid in a carboxyl group or 2-amino group which are in equilibrium with the L-2-amino-5-diaminomethyleneamino-pentanoic acid.

According to an embodiment herein, the therapeutic composition improves hair growth and muscular hypertrophy and restores the muscle mass, increases bone density, decreases fatty tissue, improves eye's central vision, decreases cellular proptosis, and improves skin elasticity

According to an embodiment herein, the therapeutic composition is synthesized from N(omega)′-protonated L-arginine through a process comprising the steps of dissolving the N(omega)′-protonated L-arginine in a liquid solvent system containing a strong acid with a known negative Hammett acidity function “H₀” to obtain a solution, bringing a first Kelvin temperature of the solution to a second Kelvin temperature, and wherein the second Kelvin temperature is less than ΔH°/(ΔS°−R(H₀−2)), and wherein the R is gas constant, and wherein the ΔH° is a change in enthalpy and wherein the ΔS° is a change in entropy change under a standard temperature and pressure conditions and wherein the standard temperature and pressure conditions includes a temperature of 25° C. and a pressure of 1 atmospheric pressure, when a N(omega)′-protonated guanidino group of the N(omega)′-protonated L-arginine is converted to a diprotonated form of the guanidino group in water, and wherein the maximum of the second Kelvin temperature is increased, when a value of the H₀ is reduced, allowing derivatives of the N(omega)′-protonated L-arginine to reach an equilibrium state in the solution at the second Kelvin temperature by providing time; and wherein said time doesn't need to be increased to more than 24 hours, and bringing the acidity of the solution to a pH value of 7.0 and the temperature of the solution to 25° C., wherein the N(omega)′-protonated 1-arginine includes its ionized forms in a carboxyl or 2-amino group, which are in equilibrium with the N(omega)′-protonated 1-arginine.

According to an embodiment herein, the therapeutic composition stimulates a synthesis and a secretion of IGF-1

According to an embodiment herein, a therapeutic compound formulated at pH 5-9 is provided. the therapeutic compound has a formula

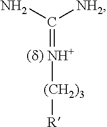

wherein the R′ is a chemical group bonding to the —(CH₂)₃— through a single C—C bond, and wherein the R′ contains a carboxyl group or an ionized form of the carboxyl group, and wherein the R′ contains an amino group or an ionized form of the amino group, and wherein the therapeutic compound has a molecular mass of more than or equal to 174.2 u.

According to an embodiment herein, the therapeutic compound is derived from a composition having a N(omega)′-protonated guanidino group through a method comprising the steps of dissolving the composition in a liquid solvent system containing a strong acid with a known negative Hammett acidity function “H₀” to obtain a solution, bringing a first Kelvin temperature of the solution to a second Kelvin temperature, and wherein the second Kelvin temperature is less than ΔH°/(ΔS−R(H₀−2)), and wherein the R is gas constant, and wherein the ΔH° is a change in enthalpy and wherein the ΔS° is a change in entropy change under a standard temperature and pressure conditions and wherein the standard temperature and pressure conditions includes a temperature of 25° C. and a pressure of 1 atmospheric pressure, when the N(omega)′-protonated guanidino group is converted to a diprotonated form of the guanidino group in water, and wherein the maximum of the second Kelvin temperature is increased, when a value of the H₀ is reduced, allowing derivatives of the N(omega)′-protonated guanidino group to reach an equilibrium state in the solution at the second Kelvin temperature by providing time; and wherein said time doesn't need to be increased to more than 24 hours, and bringing the acidity of the solution to a pH value of 7.0 and the temperature of the solution to 25° C., wherein the composition has a structural formula

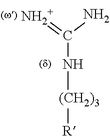

According to an embodiment herein, the therapeutic compound is derived from a composition having a N(omega)′-protonated guanidino group through a method comprising the steps of increasing a temperature of an aqueous solution containing the composition having the N(omega)′-protonated guanidino group and/or decreasing an acidity of the aqueous solution containing the composition having the N(omega)′-protonated guanidino group in structure to obtain a pH which is more than a value of pKa−2, and wherein the pKa is a minus logarithm of an acid dissociation constant of the N(omega)′-protonated guanidino group in the aqueous solution at its temperature, wherein the pKa−2 is reduced when the temperature is increased; giving time to derivatives of the N(omega)′-protonated guanidino group to reach an equilibrium state in the aqueous solution at the pH which is more than the value of pKa−2; and wherein said time doesn't need to be increased to more than 24 hours, and bringing the temperature of the aqueous solution to 25° C. and the acidity of the aqueous solution to a pH value of 7.0 after reaching the equilibrium state, and wherein the composition has a chemical structure represented by

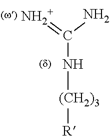

According to an embodiment herein, the therapeutic compound stimulates a synthesis and a secretion of IGF-1

According to an embodiment herein, a method of converting a composition having a N(omega)′-protonated guanidino group to a product having a N(delta)-protonated diaminomethyleneamino group is provided. The method comprises the steps of bringing a first Kelvin temperature of an aqueous solution containing the composition having the N(omega)′-protonated guanidino group to a second Kelvin temperature, and wherein the second Kelvin temperature is less than ΔH°/(ΔS°+2R), and wherein the ΔH° is a change in enthalpy and wherein the ΔS° is a change in entropy under a standard temperature and pressure conditions and wherein the standard temperature and pressure conditions include a temperature of 25° C. and a pressure of 1 atmospheric pressure, when the N(omega)′-protonated guanidino group is converted to a diprotonated form of the guanidine group in the aqueous solution, and wherein the R is gas constant; increasing an acidity of the aqueous solution containing the composition having the N(omega)′-protonated guanidino group to obtain a pH which is less than a value of pKa+2, and wherein the pKa is a minus logarithm of a first acid dissociation constant of the diprotonated form of the guanidino group of the composition at the second Kelvin temperature in the aqueous solution, and wherein the pKa+2 value is increased when the second Kelvin temperature is reduced; allowing derivatives of the N(omega)′-protonated guanidino group to reach an equilibrium state in the aqueous solution at the pH which is less than the value of pKa+2 together and at the second Kelvin temperature by providing time; and wherein said time doesn't need to be increased to more than 24 hours, and bringing the acidity of the aqueous solution to a pH value of 7.0 and the temperature of the aqueous solution to 25° C., wherein the composition has a formula represented by

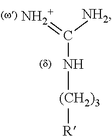

wherein the R′ is a chemical group bonding to the —(CH₂)₃— through a single C—C bond, and wherein the R′ contains a carboxyl group or an ionized form of the carboxyl group, and wherein the R′ contains an amino group or an ionized form of the amino group, and wherein the composition has a molecular mass of more than or equal to 174.2 u.

According to an embodiment herein, the N(delta)-protonated diaminomethyleneamino group stimulates a synthesis of IGF-1mRNA and IGF-1 in the cell.

According to an embodiment herein, the product is a therapeutic composition

The embodiments herein provide an ex-vivo synthesis of a biomolecule found in-vivo and involved in the signaling and stimulation of the synthesis and secretion of autocrine-paracrine Insulin like Growth Factor-I (IGF-I) in different tissues in the body.

It was noticed that any kind of exercises such as jogging, running, bicycling, walking or skating reduced the body fat mass and increased the lean muscle mass. During the exercise, blood glucose is utilized by the body cells leading to the decrease in plasma/blood glucose, which in turn induced the growth hormone (GH) production thereby stimulating autocrine-paracrine IGF-1 signaling leading to the decrease in fat tissue by lipolysis, and strengthening and development of muscles-myogenesis. These changes effectively occurred after a delay from the time when the exercise is started and continued. In addition, there is a decrease in these changes after a delay from the time when the exercise is stopped. In other words, during the course of the exercise, the above changes were not a gradual process from the beginning. This is because of the accumulation of the ‘factor’ responsible for inducing the changes in the cell at first. Thus the accumulated factor stimulates the production of autocrine-paracine IGF-1.

The Growth hormone harbors the above factor which is responsible for production of autocrine-paracine IGF-1. The Growth hormone is broken down into its respective aminoacids within the cell after interaction with its receptor, wherein the factor is formed from one of the amino acids of GH, which begins to accumulate in the cell, and then stimulating the synthesis and secretion of IGF-1.

It is noticed that in a premature neonates with very low birth weight (VLBW), when fed with dried milk containing casein, are infected with late metabolic acidosis. In this condition, inspite of feeding milk, weight gain and increase in fatty tissue does not happen due to the increase in urea. This indicates that urea causes the decrease in fatty tissue.

When we consider/assume that the stimulating of the secretion of autocrine-paracrine IGF-1 by urea, is the reason for the decrease in fatty tissue, the ‘Factor’ stimulating the synthesis and secretion of IGF-1 should have the following moiety in its chemical structure without resonance (mesomerism):

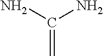

Since the urea under physiologic condition of the body (pH 7.40 and 37° C.) significantly has a chemical formula as mentioned below

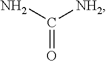

which is one of its tautomeric forms
and with regard to a pKa of 0.18 at 25° C. in protonated urea with endothermic deprotonation, that undergoing resonance(mesomerism) isn't significantly observed under physiologic condition of the body (pH 7.40 and 37° C.). Thus, the protontated form of urea can't stimulate the synthesis and secretion of IGF-1. However the length of the C—NH₂ bond equals 1.47 Å (Angstrom) in the chemical formula.

Since, the ‘Factor’ is one of the amino acids of GH, next step of the embodiments herein is to examine the amino acid of GH having the following moiety in its chemical structure without resonance (mesomerism).

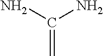

In addition to the above, the ‘Factor’ wouldn't join its intracellular receptor to stimulate the synthesis and secretion of IGF-1 when the length of the C—NH₂ bond didn't equal 1.47 Å (Angstrom) in the moiety. It's clear the resonance makes the length shorter than 1.47 Å.

It was found that none of the amino acids of extracellular GH before joining its receptor on the cell surface had the above shown moiety, but it was observed that if the double bond in the N(omega)′-protonated Guanidino group (NH₂—C(═NH₂⁺)—NH—) of N(omega)′-protonated L-arginine which is one of the amino acids of extracellular GH changes its place, then new bio-molecule having the following moiety in its chemical structure is possible. (Guanidino=diaminomethylideneamino)

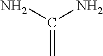

This new biomolecule having the above moiety in its structure is referred for this invention as N(delta)-protonated L-2-amino-5-diaminomethyleneamino-pentanoic acid or ‘J-Factor’. The diagrams shown below indicate the transition of the double bond in L-arginine to J-factor:

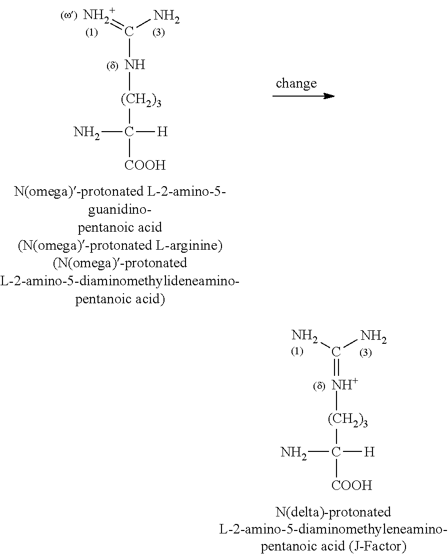

“Advanced Organic Chemistry” (2004, 4th edition) by Francis A. Carey and Richard J. Sundberg on page 9 discloses: “In most cases, the delocalization of electrons as represented by the writing of alternative Lewis structures is associated with enhanced stability relative to a single localized structure. This is not always true, however, since molecules and ions are known in which electron delocalization produces an increase in energy relative to a localized model.” Thus, absence of resonance in the side-chain of N(omega)′-protonated L-arginine and J-Factor isn't impossible.

Regarding the significant difference of the standard enthalpy changes in the following reactions:

L-arginine_{(aq)}+H⁺_{(aq)}→N(omega)′-protonated L-arginine_{(aq)}+12.3 kcal/mol

Guanidine_{(aq)}+H⁺_{(aq)}→Guanidinium_{(aq)}+33 kcal/mol

it's clear that resonance isn't observed in the side-chain of N(omega)′-protonated L-arginine because resonance significantly decreases the energy level of Guanidinium, in contrast to N(omega)′-protonated L-arginine.

After a dissolution of the cyrstalline N(omega)′-protonated Larginine in water, 15N NMR studies indicate the equivalence of the N₁ and N₃ chemical shifts and therefore, fast rotation (angular speed) about the C—N_{(δ)} bond. Consequently, the C—N_{(δ)} bond of N(omega)′-protonated Larginine is single and don't participate in resonance (mesomerism).

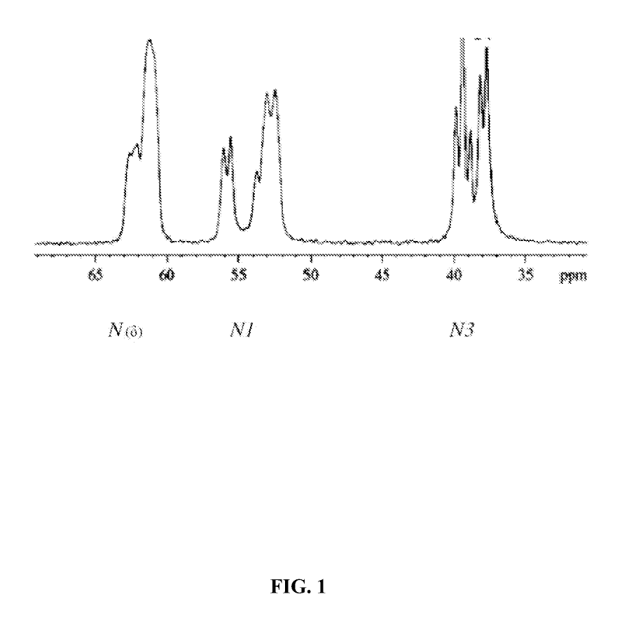

With respect to FIG. 1, 15N NMR spectra of cyrstalline L-arginine hydrochloride (The Journal of Physical Chemistry B, page 15416) indicate an absence of resonance (mesomerism) between the C—N₁, C—N₃ and C—N_{(δ)} bonds of N(omega)′-protonated L-arginine, If the bonds had resonance (mesomerism), regarding the number“78”, the formula of N(omega)′-protonated L-arginine would be as follows:

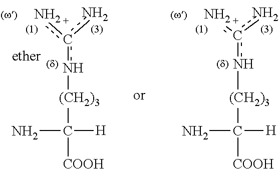

So the pair of nuclei of the N₁ and N₃ would be interchangeable by rotation about C—N_{(δ)} as an axis of symmetry. Consequently, the nuclei would be chemically equivalent and have the same chemical shift, while according to the 15N NMR spectra in FIG. 1, their chemical shifts aren't the same.

Therefore, based on Reductio ad absurdum the resonance (mesomerism) don't exist and the exact formula of N(omega)′-protonated L-arginine is as follows:

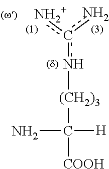

On the other hand, neither of the C—NH₂ bonds of J-Factor in the side-chain is involved by the electron cloud of resonance because the kinetic energy related to the C—N_{(δ)} bond rotation changed to that related to the rotation of the C—NH₂ bonds, when N(omega)′-protonated L-arginine is changed to J-Factor. In other words, a rapid rotation involves the C—NH₂ bonds of J-Factor and indicates they don't have resonance (mesomerism).

In fact, both of the groups (NH₂) moving around the C—N_{(δ)} axis in N(omega)′-protonated L-arginine have an initial level of the kinetic energy and linear speed. After changing to J-Factor, the groups will have at least the previous level of the kinetic energy and consequently, with regard to Eₖ=1/2 mv² (The kinetic energy is equal to the mass multiplied by the square of the linear speed), the groups will have at least the previous linear average speed around the C—NH₂ axis with a smaller radius. In other words, the angular speed (rotation) about the C—NH₂ bond of J-Factor is more rapid than that about the C—N_{(δ)} bond of N(omega)′-protonated L-arginine which is single and without resonance (mesomerism). Therefore, the C—N_{(δ)} and C—NH₂ bonds are respectively double and single in J-Factor.

Anyway, N(omega)′-protonated L-arginine and J-Factor aren't chemically the same even if you suppose J-Factor would have mesomerism in its side-chain unlike N(omega)′-protonated L-arginine.

Hence it's reasonable that at least one of the N(omega)′-protonated L-arginines existing in the structure of extracellular GH changes into J-Factor to be able to stimulate the synthesis and secretion of IGF-1.

The extracellular GH does not have J-Factor in its structure. Consequently, the change should be done after GH joins its receptor and before it's broken down into its amino acids in the cells. if the N(omega)′-protonated L-arginine changed after the breaking down of GH, the other N(omega)′-protonated L-arginine biomolecules not existing in GH should be considered. Accordingly the receptor of GH changes at least one of the N(omega)′-protonated L-arginines of GH into J-Factor by enzymatic action and finally, after GH is broken down into its amino acids, J-Factor accumulates in the cells and stimulates autocrine-paracrine IGF-1 after the moiety bonds with its intracellular receptor. Clearly, any composition having the moiety without resonance (mesomerism) stimulates the IGF-1 after reaching the minimum of the effective molar concentration in the cell, because the moiety is the agent of the stimulation through bonding with its receptor. On the other hand, any factor to increase the tendency of the moiety to its receptor declines the minimum of the concentration. For example, in J-Factor, the proton bonding with N(delta) makes an ionic bond with the negative charge next to the receptor, while the moiety is attached to its receptor by the Van der Waals force that is very much weaker than the ionic bond, so that the minimum molar concentration of J-Factor stimulating the IGF-1 in the cell is very much less than L-2-amino-5-diaminomethyleneamino-pentanoic acid. Therefore, J-factor is a strong stimulator for the synthesis and secretion of autocrine paracrine IGF-1 whereas L-2-amino-5-diaminomethyleneamino-pentanoic acid is an extremely weak stimulator for it.

Anyway, the carboxyl and 2-amino groups of J-Factor, N(omega)′-protonated L-arginine, L-2-amino-5-diaminomethyleneamino-pentanoic acid and the guanidino form of L-arginine are in equilibrium with their ionized forms. Thus, J-Factor consists of N (delta)-protonated L-2-amino-5-diaminomethyleneamino-pentanoic acid and its ionized forms in the carboxyl or 2-amino groups which are in equilibrium with it. The above fact also holds good for N(omega)′-protonated L-arginine, L-2-amino-5-diaminomethyleneamino-pentanoic acid and the guanidino form of L-arginine and any other alpha-amino acid.

With regard to the data mentioned above, N(delta)-protonated L-2-amino-5-diaminomethyleneamino-pentanoic acid, N(omega)′-protonated L-arginine, the two other above molecules or any other alpha-amino acid are mentioned anywhere even as a structural formula, their ionized forms in the carboxyl or 2-amino group which are in equilibrium with them are also considered. (2-amino group=alpha-amino group).

On the other hand, with respect to N(omega)′-protonated 1-arginine and J-Factor, the value of pKa=12.48 at room temperature (25° C.) and the value of pKa=11.68 at 37° C. in the guanidinium and N(delta)-protonated diaminomethyleneamino groups and the henderson-hasselbalch equation (log([A−]/[HA])=pH−pKa), at pH less than 7.48, for any 100000 and 15848(=10^{11.68-7.48=4.20}) molecules respectively at 25° C. and 37° C., there is less then one biomolecule in deprotonated form in the side chain. Furthermore, the deprotonation of the guanidinium and N(delta)-protonated diaminomethyleneamino groups is endothermic and reversible. Therefore the results are as follows.
1—At pH less than 7.48 together with 25° C. or less, the concentration of their deprotonated form in the side chain is too little and insignificant to be chemically considered.
2—Under physiological conditions (pH 7.40 and 37° C.) of a living body, the concentration of L-2-amino-5-diaminomethyleneamino-pentanoic acid not protonated in the side chain and a extremely weak stimulator for autocrine-paracrine IGF-1, is too little and insignificant to stimulates synthesis and secretion of the IGF-1.
3—At pH less than 7.48 together with 25° C. or less and under physiological conditions (pH 7.40 and 37° C.), 1-arginine and L-2-amino-5-diaminomethyleneamino-pentanoic acid is practically as protonated in the side chain.

Furthermore, J-Factor has pKa's of 2.17, 9.04 and 12.48 respectively in carboxyl, protonated 2-amino and N(delta)-protonated diaminomethyleneamino groups at room temperature (25° C.) and the deprotonation of the groups is endothermic and reversible. In addition, it has a Pka of 8.39 in the protonated 2-amino group at 37° C. calculated based on the integrated form of the van't Hoff equation and ΔH°=43.8 kj/mol=10.0 kcal/mol in the deprotonation of the protonated 2-amino group. Consequently, with respect to/regarding the Henderson-Hasselbalch equation, the formula of the J Factor is as follows:
1—At 25° C., the dominant structural formula of J-Factor at pH more than 2.17 and less than 9.04 is as follows:

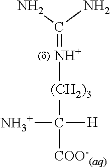

2—Under physiological conditions (pH 7.40 and 37° C.) of a living body, J-Factor is dominantly as the formula.
2—At pH 7.0 together with 25° C., J-Factor is approximately as the formula.

### EXPERIMENTS & RESULTS

According to an embodiment of the embodiments herein, N(omega)′-protonated L-arginine was chemically converted into J-Factor ex-vivo. To prevent synthetic J-factor from damaging the alimentary canal after oral administration, it was formulated at a pH value that is safe for the canal like 7.0 which is the safest pH. Then, converted/synthetic produced ex-vivo J-Factor was orally administrated to a small group of 11 volunteers for one year. Each volunteers were given 0.5 mg per 10 days (=1.5 mg/month) and photographs were taken in each case to record the changes in their faces and heads.

In addition, to eliminate other factors probably influencing results during the trial, they were prevented from exercising and changing previous food schedule such as meat except the J-Factor. Their intake of meat was at least 200 gram per day in the food schedule before the trial and wouldn't change during the trial. Because the amount of N(omega)′-protonated L-arginine in raw meat is more than 1%, their intake of N(omega)′-protonated L-arginine was more than 60000 mg per month through meat without change before and during the trial, which was more than 40000 times the J-Factor intake. Anyway, one-third of N (omega)′-protonated L-arginine is chemically converted into J-Factor and they can't separated from each other. Thus, regardless of food L-arginine, Each volunteer took N(omega)′-protonated L-arginine 3 mg per month together with J-Factor that was less than 1/20000 times the N(omega)′-protonated L-arginine intake through meat without change before and during the trial. Therefore, the physical changes observed in the volunteers weren't caused by N(omega)′-protonated L-arginine.

Anyway, the triceps skin fold (TSF) thickness was measured in millimetres with the caplier at the mid-back of the right mid-upper arm [mid-point between the tip of the shoulder and the tip of the elbow (olecranon process and the acromium)] to evaluate the change in fatty tissue during the trial. The amounts measured and calculated are listed below:

TSF (mm)

| Name | Sex | Age | Start | End | Decrease |
|---|---|---|---|---|---|
| Mahshid | ♀ | 18 y | 15 | 10 | 5 |
| Elnaz | ♀ | 19 y | 19 | 12 | 7 |
| Elham | ♀ | 26 y | 14 | 10 | 4 |
| Maryam | ♀ | 30 y | 21 | 14 | 7 |
| Fatemeh | ♀ | 31 y | 20 | 13 | 7 |
| Farzaneh | ♀ | 43 y | 36 | 17 | 19 |
| Akhtar | ♀ | 43 y | 18 | 13 | 5 |
| Hamideh | ♀ | 43 y | 38 | 20 | 18 |
| Masoud | ♂ | 38 y | 6 | 3 | 3 |
| Mahmood | ♂ | 47 y | 7 | 3 | 4 |
| Mohammad Ali | ♂ | 49 y | 13 | 7 | 6 |

| | | | |
|---|---|---|---|
| sum | 207 | 122 | 85 |
| | | | (41.7%) |

1-Start = Just before starting the trial
2-End = one year after the start.
The above data show a decrease in fatty tissue by J-Factor.

To evaluate muscular hypertrophy, the mid-upper arm muscle circumference (MUAMC) was calculated from the right arm at the beginning and end of the trial. It's derived in cm from the MUAC (mid-upper arm circumference) in cm and the TSF (triceps skin fold) in mm according to the formula “MUAMC=MUAC−(π×TSF/10)”. Finally, the following numbers were measured and concluded:

MUAC (cm)

| Name | Sex | Age | Start | End |
|---|---|---|---|---|
| Mahshid | ♀ | 18 y | 23.5 | 23.0 |
| Elnaz | ♀ | 19 y | 31.0 | 31.5 |
| Elham | ♀ | 26 y | 29.5 | 30.0 |
| Maryam | ♀ | 30 y | 31.0 | 31.6 |
| Fatemeh | ♀ | 31 y | 28.0 | 28.2 |
| Farzaneh | ♀ | 43 y | 33.2 | 29.3 |
| Akhtar | ♀ | 43 y | 25.2 | 25.9 |
| Hamideh | ♀ | 43 y | 31.4 | 27.5 |
| Masoud | ♂ | 38 y | 25.3 | 25.6 |
| Mahmood | ♂ | 47 y | 28.3 | 29.0 |
| Mohammad Ali | ♂ | 49 y | 31.5 | 31.7 |

1-Start = Just before starting the trial
2-End = one year after the start

| | | | MUAMC (cm) | | |
|---|---|---|---|---|---|
| Name | Sex | Age | Start | End | increase |
| Mahshid | ♀ | 18 y | 18.8 | 19.9 | 1.1 |
| Elnaz | ♀ | 19 y | 25.0 | 27.7 | 2.7 |
| Elham | ♀ | 26 y | 25.1 | 26.9 | 1.8 |
| Maryam | ♀ | 30 y | 24.4 | 27.2 | 2.8 |
| Fatemeh | ♀ | 31 y | 21.7 | 24.1 | 2.4 |
| Farzaneh | ♀ | 43 y | 21.9 | 24.0 | 2.1 |
| Akhtar | ♀ | 43 y | 19.6 | 21.6 | 2.0 |
| Hamideh | ♀ | 43 y | 19.5 | 21.2 | 1.7 |
| Masoud | ♂ | 38 y | 23.4 | 24.7 | 1.3 |
| Mahmood | ♂ | 47 y | 26.1 | 28.1 | 2.0 |
| Mohammad Ali | ♂ | 49 y | 27.4 | 29.5 | 2.1 |

| | | | |
|---|---|---|---|
| sum | 252.9 | 274.9 | 22.0 |
| | | | (8.7%) |

1-Start = Just before starting the trial
2-End = one year after the start.
The above data show muscular hypertrophy by J-Factor

Eye' central vision was examined with the E-chart and the following numbers were measured and concluded:

Right Eye' central vision

| Name | Sex | Age | Start | End | increase |
|---|---|---|---|---|---|
| Mahshid | ♀ | 18 y | 8 | 10 | 2 |
| Elnaz | ♀ | 19 y | 9 | 10 | 1 |
| Elham | ♀ | 26 y | 10 | 10 | ZERO |
| Maryam | ♀ | 30 y | 2 | 6 | 4 |
| Fatemeh | ♀ | 31 y | 1 | 3 | 2 |
| Farzaneh | ♀ | 43 y | 8 | 10 | 2 |
| Akhtar | ♀ | 43 y | 8 | 10 | 2 |
| Hamideh | ♀ | 43 y | 10 | 10 | ZERO |
| Masoud | ♂ | 38 y | 10 | 10 | ZERO |
| Mahmood | ♂ | 47 y | 9 | 10 | 1 |
| Mohammad Ali | ♂ | 49 y | 5 | 8 | 3 |

1-Start = Just before starting the trial
2-End = one year after the start.
The above data show improvement in eye's central vision by J-Factor.

Left Eye' central vision

| Name | Sex | Age | Start | End | increase |
|---|---|---|---|---|---|
| Mahshid | ♀ | 18 y | 9 | 10 | 1 |
| Elnaz | ♀ | 19 y | 9 | 10 | 1 |
| Elham | ♀ | 26 y | 10 | 10 | ZERO |
| Maryam | ♀ | 30 y | 1 | 3 | 2 |
| Fatemeh | ♀ | 31 y | 3 | 5 | 2 |
| Farzaneh | ♀ | 43 y | 9 | 10 | 1 |
| Akhtar | ♀ | 43 y | 7 | 10 | 3 |
| Hamideh | ♀ | 43 y | 10 | 10 | ZERO |
| Masoud | ♂ | 38 y | 9 | 10 | 1 |
| Mahmood | ♂ | 47 y | 9 | 10 | 9 |
| Mohammad Ali | ♂ | 49 y | 6 | 9 | 3 |

1-Start = Just before starting the trial
2-End = one year after the start.
The above data show improvement in eye's central vision by J-Factor.

It's clear that any composition that stimulates the synthesis and secretion of autocrine-paracrine IGF-1 performs it only through increasing the expression of IGF-1mRNA in the cell. Therefore, the expression of IGF-1mRNA was measured in the fatty tissue at the mid-back of the mid-upperarm of the volunteers by real-time RT-PCR (Reverse transcription-polymerase chain reaction) at the beginning and end of the trial and the following numbers were obtained:

| | | | IGF-1 mRNA (copy/mg) | | |
|---|---|---|---|---|---|
| Name | Sex | Age | Start | End | increase |
| Mahshid | ♀ | 18 y | 3.8 × 10⁶ | 7.2 × 10⁸ | yes |
| Elnaz | ♀ | 19 y | 2.7 × 10⁶ | 6.0 × 10⁸ | yes |
| Elham | ♀ | 26 y | 1.7 × 10⁵ | 2.1 × 10⁹ | yes |
| Maryam | ♀ | 30 y | 2.2 × 10⁴ | 1.0 × 10¹⁰ | yes |
| Fatemeh | ♀ | 31 y | 4.3 × 10⁴ | 5.2 × 10⁷ | yes |
| Farzaneh | ♀ | 43 y | 2.6 × 10³ | 6.3 × 10⁶ | yes |
| Akhtar | ♀ | 43 y | 8.3 × 10² | 1.1 × 10⁹ | yes |
| Hamideh | ♀ | 43 y | 2.4 × 10³ | 1.8 × 10⁷ | yes |
| Masoud | ♂ | 38 y | 8.3 × 10² | 7.7 × 10⁷ | yes |
| Mahmood | ♂ | 47 y | 4.5 × 10⁵ | 1.2 × 10¹⁰ | yes |
| Mohammad AM | ♂ | 49 y | 9.4 × 10² | 9.2 × 10⁶ | yes |

1-Start = Just before starting the trial
2-End = one year after the start.
The above data show a increase in the expression of IGF-1mRNA and consequently the stimulation of the synthesis and seceretion of autocrine-paracrine IGF-1 by J-Factor.

Thus, the following physical changes were observed in case of each voluntary after one year. The physical changes include a hair growth; muscular hypertrophy; a decrease in fatty tissue e.g. in abdomen; improvement in eye's central vision; and improved tone and color of facial skin. These changes indicate that ex-vivo synthesized J-Factor functioned like GH and autocrine-paracrine IGF-1 and also stimulated IGF-1 production.

After the first year, the applicant withdrew J-Factor and would give the initial N(omega)′protonated L-arginine (reactant) not converted to J-Factor 1.5 mg per 10 days to each volunteer without any other changes and wouldn't make them aware, in order to better compare the reactant and J-Factor. During the second year (the second trial), the physical changes would return. Therefore,
1—J-Factor and N(omega)′-protonated L-arginine aren't chemically the same and resonance (mesomerism) isn't observed in their side-chain
2—N(omega)′-protonated L-arginine don't stimulated IGF-1 production unlike ex-vivo synthesized J-Factor
3—initial N(omega)′-protonated L-arginine doesn't mix with J-Factor
4—The product derived from N(omega)′-protonated L-arginine through the methods of this invention stimulates the synthesis and secretion of IGF-1 and has therapeutic effects caused by said IGF-1 even if you suppose said product would be either no J-Factor or J-Factor with mesomerism in its side-chain unlike N(omega)′-protonated L-arginine.

The mechanism that takes place after GH interacts with its receptor is, at least one of its N(omega)′-protonated L-arginines is changed to J-Factor by the enzymatic interaction with the receptor. GH is broken down into its respective amino acid units within the cell, thus allowing accumulation of J-Factor gradually and to stimulate the secretion of autocrine-paracrine IGF-I.

### Example 1

### Use of the Invention or Synthetic J-Factor

Based on the above results it is found that synthetic J-Factor accumulates in the body's different cells and stimulates the synthesis and secretion of autocrine-paracrine IGF-1, after an oral administration.

Consequently, it could have anti-aging effect and the following effects are found. The uses/effects are the same as the autocrine-paracrine IGF-1 functions in the body which includes but not limited to muscular hypertrophy and restoring the muscle mass, thus giving a toned up and youthful look; increase in bone density, to avoid bone fracture which is common in old people; and decrease in fatty tissue, to avoid obesity-related diseases such as diabetic, cardiovascular problems etc., improvement in eye's central vision; hair growth; decrease in cellular proptosis; improvement in skin elasticity and other IGF-1 effects.

In fact, all of the above mentioned changes help in preventing or limiting the aging signs. The advantage of using ex-vivo synthesized J-Factor of the embodiments herein when compared to biosynthetic GH is shown in the comparative table below:

| Synthetic J-Factor | Biosynthetic GH |
|---|---|
| 1- Oral administration | Intra muscular (IM) |
| 2- through injection | administration |
| 3 times a month | 3 time a week |
| Non-diabetogenic | Diabetogenic |
| Non-antigenic | Antigenic |
| Easily synthesized | Biosynthetic |
| Synthetic J-Factor of the present | Complications: |
| invention showed no complications | Joint swelling Carpal tunnel |
| such as Joint swelling, Carpal | syndrome and Joint pain |
| tunnel syndrome or Joint pain | |
| in the 11 volunteers which are | |
| common while using biosynthetic GH. | |

With regards to the above mentioned data, the following result is obtained:

In J-Factor, the N(delta)-protonated diaminomethyleneamino group stimulates the synthesis and secretion of autocrine-paracrine IGF-1. Thus, a J-Factor analogue stimulates the synthesis and secretion of IGF-1 and is a therapeutic compound due to having the group without resonance. (the J-Factor analogue is defined below)

Furthermore, protonated arginyl residues in proteins don't have resonance (mesomerim) as observed in N(omega)′-protonated L-arginine and J-Factor, unlike guanidinium and methyleguanidinium.

In fact, when you give a constant kinetic energy through dissolution, heat and so on to the following formulas in cyrstalline form, based on the law of inertia, the parts with less

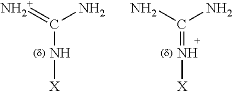

rotational inertia, receive more angular speed (rotation) compared to the parts with more rotational inertia.

Therefore, the higher the rotational inertia of the substituent “X”, the higher the ratio of the angular speed (rotation) of the other parts to that of the substituent “X” and consequently, less likely the resonance (mesomerim). Absence of resonance in protonated arginyl residues of proteins unlike guanidinium and methyleguanidinium confirms the above truth.

Further, the resonance isn't observed in N(omega)′-protonated Larginine and J-Factor Therefore, when the rotational inertia is more than or equal to that of the “X” in L-arginine and J-Factor, the resonance doesn't exist in N(delta)-protonated diaminomethyleneamino and N(omega)′-protonated guanidino groups, regardless of the resonance or inductive effect of the “X” We know that rotational inertia is calculated through the following equation:

I=ΣI₁=Σm₁r₁²=m₁r₁²+m₂r₂²+m₃r₃²+ . . .

wherein I is total rotational inertia, Ii is rotational inertia of a particle, r₁ is the distance of the partical from the axis and m₁ is the mass of the particle.

With respect to the following formulas with molecular mass more than or equal to the minimum molecular mass of N(omega)′-protonated L-arginine and J-Factor (174.2 u)

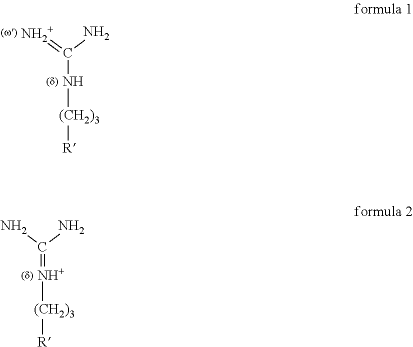

wherein R′ of the above formulas is a chemical group bonding to —(CH₂)₃— through a single C—C bond, wherein the R′ contains a carboxyl group or its ionized form, wherein the R′ contains an amino group or its ionized form,
it's clear from the above that under the above limitations, one can increase the mass of the substituent “—(CH₂)₃—R′” in the formulas to a value more than mass of the substituent in N(omega)′-protonated L-arginine and J-factor only through an increase in the number of atoms (particles) except for one case. In other words, new m₁r₁² s are added to the previous Σm₁r₁² and total rotational inertia (I) of the substituent” “—(CH₂)₃—R′” increases. In said one case, the hydrogen bonding to the alpha carbon in N(omega)′-protonated L-arginine and J-factor is substituted with an atom having more mass and consequently, more rotational inertia. Therefore, under the above limitations, the total rotational inertia (I) of the substituent “—(CH₂)₃—R′” is more than or equal to that of the substituent in N(omega)′-protonated L-arginine and J-factor.
Consequently, with regard to
1—Absence of resonance effect in the substituent “—(CH₂)₃—R′”
2—Brief change in the inductive effect of the substituent “—(CH₂)₃—R′” in the spite of the “R′” change, due to the fact that —(CH₂)₃— is long, wherein if the R′ bonds to —(CH2)₃— through a single C—C bond, the brief change is less.
3—The fact that the higher rotational inertia of the substituent, less likely is the resonance, regardless of the resonance and inductive effects of the substituent.
4—Absence of resonance in the side-chain of N(omega)′-protonated Larginine and J-factor
5—their minimum mulecular mass of 174.2 u
   neither of the compositions having one of the following formulas together with molecular mass more than or equal to 174.2 u has resonance (mesomerism) in N(omega)′-protonated guanidino and N(delta)-protonated diaminomethyleneamino groups,

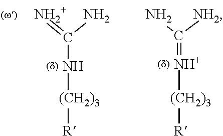

wherein R′ of the above formulas is a chemical group bonding to —(CH₂)₃— through a single C—C bond, wherein the R′ contains a carboxyl group or its ionized form, and wherein the R′ contains an amino group or its ionized form.
In other words, they aren't the same.

Thus, a composition having molecular mass more than or equal to 174.2 u and the N(delta)-protonated diaminomethyleneamino group in its chemical structure without resonance or mesomerism stimulates the synthesis and secretion of autocrine paracrine IGF-1 in tissues in a living body and have the therapeutic effects of J-Factor mentioned above, wherein the composition has a formula,

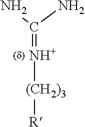

wherein R′ is a chemical group bonding to —(CH₂)₃— through a single C—C bond, wherein the R′ contains a carboxyl group or its ionized form, wherein the R′ contains an amino group or its ionized form, wherein the composition is therapeutic, wherein the composition is called a J-Factor analogue.

In fact, the N(delta)-protonated diaminomethyleneamino group without resonance(mesomerism) in a composition like J-Factor stimulates the synthesis and secretion of IGF-1 by increasing the expression of IGF-1mRNA in all of the cells of a living body. In other words, the group without resonance(mesomerism) stimulates the synthesis of IGF-1 mRNA and consequently IGF-1 in the cell.

Furthermore, in this application a chemical group is two or more atoms bound together as a single unit and forms part of a molecule or ion and its atoms can have electrical charge.

Now, before explaining the methods of synthesizing the J-Factor and its analogues ex-vivo, the Hammett acidity function(H₀), pH and strong acids are explained below:
1—The pH scale ranges from 0 to 14 and is measured by the pH meter or indicator.
2—The Hammett acidity function (H₀) is a measure of acidity that is used for very concentrated solutions of strong acids, including superacids and the best-known acidity function used to extend the measure of Bronsted-Lowry acidity beyond the dilute aqueous solutions (pH 0-14) for which the pH scale is useful. In the other hand, in this application except for Examples 5 and 6, dilute aqueous solutions (pH 0-14) are only used. Thus, the Hammett acidity function (H₀) isn't needed here except for Examples 5 and 6 unlike the pH, which is the negative logarithm of the hydrogen ion molarity (pH=−log [H+]). In addition, hydrogen ions exist as the hydronium ion (H₃O⁺) in dilute aqueous solutions (pH 0-14), not alone, Thus, pH=−log [H₃O⁺]=−log [H⁺] in them. (An aqueous solution is a solution in which the solvent is water. It is usually shown in chemical equations by appending (aq) to the relevant formula).
3—The Hammett acidity function (H₀) extends the measure of Brønsted-Lowry acidity beyond a pH value of zero as shown in the Tables 1 and 2 of this application.
4—The Hammett acidity function are defined in terms of a buffered medium containing a weak base B and its conjugate acid BH⁺:

H₀=pKa+log([B]/[BH⁺])
where pKa is the negative logarithm of the dissociation constant of BH⁺ in water.
5—According to the classical definition superacid is an acid with an acidity greater than that of 100% pure sulfuric acid, which has a Hammett acidity function (H₀) of −11.93. According to the modern definition, superacid is a medium, in which the chemical potential of the proton is higher than in pure sulfuric acid (higher than a H₀ value of −11.93). The value H₀=−11.93 for pure sulfuric acid must not be interpreted as pH=−11.93 (which would imply an impossibly high H₃O⁺ concentration of 10^{+11.93} mol/L in ideal solution). Instead it means that the acid species present (H₃SO₄⁺) has a protonating ability equivalent to H₃O⁺ at a fictitious (ideal) concentration of 10^{11.93} mol/L, as measured by its ability to protonate weak bases. Anyway, except for Examples 5 and 6, the above definition isn't needed for the dilute aqueous solutions (pH 0-14) of this invention, because in this case, H₀ is equivalent to pH values determined by Henderson-Hasselbalch equation.
6—A strong acid is one that completely ionizes (dissociates) in water. The pKa of a strong acid is practically independent on the temperature change because its standard enthalpy change of the acid dissociation in water is almost zero (slightly endothermic).
7—This is a list of strong acids with pKa<−1.74, which is stronger than hydronium ion, from strongest to weakest.
   Hydroiodic acid HI (pKa=−9.3)
   Hydrobromic acid HBr (pKa=−8.7)
   Perchloric acid HClO4 (pKa≈−8)
   Sulfuric acid H2SO4 (first dissociation only, pKa1=−6.62)
   Hydrochloric acid HCl (pKa=−6.3)
   p-Toluenesulfonic acid (pKa=−2.8)
8—Almost strong acids do not meet the strict criterion of being more acidic than H3O⁺, although in very dilute solution they dissociate almost completely, so sometimes they are included as “strong acids”
   Hydronium ion H3O⁺ (pKa=−1.74)
   Nitric acid HNO3 (pKa=−1.64)
   Chloric acid HClO3 (pKa=−1.0)
9—The H₀ values of some 100% pure strong acids are as follows: (Extremely strong acids)
   Fluoroantimonic acid: −31.3
   Magic acid: −19.2
   Carborane superacid: −18.0
   Fluorosulfuric acid: −15.1
   Triflic acid: −14.1
10—In the highly concentrated solutions of strong acids, simple equations such as the Henderson-Hasselbalch one are no longer valid due to the variations of the activity coefficients.

### Example 2

### Method of Synthesizing the J-Factor Ex-Vivo

For ex-vivo synthesis of J-Factor starting material is N(omega)′-protonated L-arginine. We can synthesize J-Factor in presence of an acid with a pH<pKa+2 as an acidic catalyst in an aqueous solution according to following reaction, wherein the pKa is greater than −2 and minus the logarithm of the first acid dissociation constant of the diprotonated form of the guanidine group of the starting material in the aqueous solution at the temperature of the reaction. (aq=aqueous solution)

The Reaction is represented as follows:

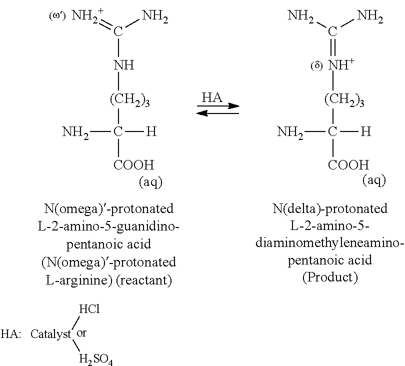

Once the chemical reaction equilibrium is established and J-Factor concentration becomes maximal in the aqueous solution at the pH<pKa+2, in order to convert all of the chemical intermediates which have a diprotonated side chain and remain after equilibrium state, to N(omega)′-protonated Larginine and J-Factor and, the acidic catalyst (HCl or H2S04) is separated from the solution by
1. using an anion exchange resin column chromatography to obtain a pH value of 7.0 together with 25° C.

R=Resin R—OH+HCl→R—Cl+H₂O

2R—OH+H₂SO₄→R₂SO₄+2H₂O

or
2. gradually dissolving Ba(OH)₂ into the solution of the H₂S0₄, while its pH value is constantly measured using a pH meter to obtain a pH value of 7.0 together with 25° C.

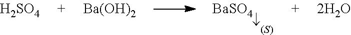

One of the qualities needed for a substance to be administrated as a drug is a suitable pH to prevent a damage to body. Furthermore, synthetic J-Factor with the pH value of about 7.0 is effective through injection in sterile condition, because finally, it reaches body cells through blood circulation.

In any case, L-2-amino-5-diaminomethyleneamino-pentanoic acid is enrichable neither at acidic pH and nor under physiological conditions unlike J-Factor.

The J-Factor synthesis mechanism: Below reaction which is reversible at any stage illustrates how the N(omega)′-protonated L-arginine is converted to J-factor at the examples 2 and 3 in ex-vivo condition in the embodiments herein:

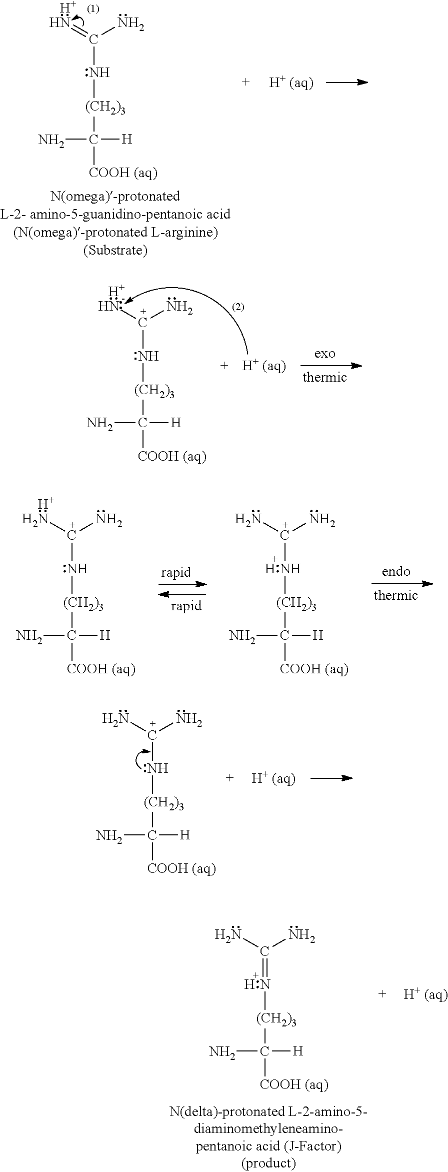

### Example 3

### The Total Method of J-Factor Synthesis Including the Above One

First, decrease or bring a first kelvin temperature of the N(omega)′-protonated L-arginine solution to a second kelvin temperature less than ΔH°/(ΔS°+2R) to obtain a pKa>−2. Second, increase the acidity of the solution to obtain a pH<the pKa+2 at which the following chemical intermediate is enriched enough

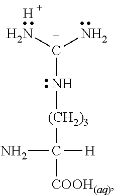

wherein the moiety

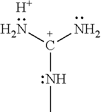

is both the diprotonted from of the guanidino group and the diprotonated guanidino group, wherein the ΔH° is change in enthalpy in a standard state (25° C. and 1 atmospheric pressure) when the moeity is derived from the N(omega)′-protonated guanidine group of the N(omega)′-protonated L-arginine, where the ΔS° is the standard entropy change (at 25° C. and 1 atmospheric pressure) when the moeity is derived from the N(omega)′-protonated guanidino group of the N(omega)′-protonated L-arginine, where the R is gas constant, wherein an increase in the acidity is preformed through adding an acid such as sulfuric acid or hydrochloric acid to the solution, wherein the pKa is a minus logarithm of the first acid dissociation constant of the diprotonted guanidino group of the above chemical intermediate in the solution at the second kelvin temperature of the solution, wherein the lower the second kelvin temperature, the higher the pKa+2.

Third, all of the derivatives of L-arginine are given time to reach an equilibrium state at the pH<the pKa+2 together with the second kelvin temperature according to the following reaction, wherein some of the initial N(omega)′-protonated L-arginine is converted to J-Factor at the pH<the pKa+2 together with the second kelvin temperature, wherein said time doesn't need to be increased to more than 24 hours:

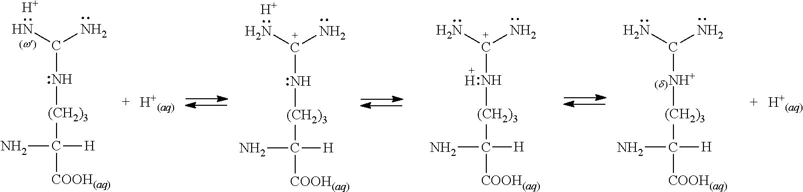

Finally, the acidity of the solution is brought to a pH 7.0 and the temperature of the solution is brought to 25° C. to convert derivatives of L-arginine which have a diprotonated side chain and remain after the equilibrium state, to N(omega)′-protonated L-arginine and J-Factor, wherein a decrease in the acidity is performed through/by adding an a base such as barium hydroxide to the solution or another way like anion exchange resin column chromatography.
According the above process, one-third of the initial N(omega)′-protonated L-arginine is converted to J-Factor with a pH 7.0 together with 25° C. as follows:

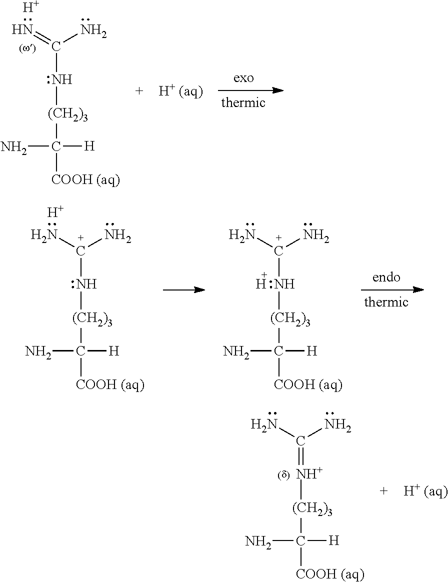

With regard to the J-Factor synthesis mechanism mentioned before the Example 3, high temperature prevents the second proton from bonding to the nitrogen of the N(omega)′-protonated guanidino group of N(omega)′-protonated L-arginine, because the bonding is exothermic and reversible. In other words, at high temperature like boiling the solution, N(omega)′-protonated L-arginine isn't covered to J-Factor through the above methods. By contrast, temperature less than 17.97° C. is favorable for the forward progress of the conversion of N(omega)′-protonated L-arginine to J-Factor through the above methods—that is, decrease in temperature accelerates forward progress of the conversion. The above mentioned result is interpreted as follows:

Based on Van't Hoff equation, the integrated form of reaction is:

Log(K₂/K₁)=(ΔH°/R)(1/T₁−1/T₂)

Wherein K₁ is the equilibrium constant at absolute temperature T₁, K₂ is the equilibrium constant at absolute temperature T₂,R=1.9858775(34)×10⁻³ kcal/(mol ° Kelvin) is gas constant and ΔH is change in enthalpy which is positive in endothermic reactions, and negative in heat-releasing exothermic processes and equal to the sum of non-mechanical work done on it and the heat supplied to it. Anyway, at constant pressure, ΔH equals the heat absorbed (or released) by a chemical reaction and in the equation, ΔH° is change in enthalpy (ΔH) in a standard state(25° C. and 1 atmospheric pressure)

Further with respect to Van't Hoff equation:

Log K=(−ΔH°/RT)+(ΔS°/R)

wherein ΔS° is the standard entropy change (at 25° C. and 1 atmosphericpressure) and K is the equilibrium constant at absolute temperature T.

Now the calculated ratios of K₂ at 100° C., 75° C., 50° C., 37° C., 30° C. to K₁ at 25° C. and others amounts in the bonding which is exothermic and reversible according to the above equations are as follows:
1—In the bonding of the second proton to the nitrogen of the N(omega)′-protonated guanidino group of N(omega)′-protonated L-arginine, pKa=−Log 1/K=log K, wherein the pKa is minus the logarithm of the first acid dissociation constant of the diprotonted guanidino group of the following chemical intermediate:

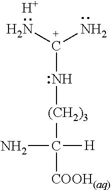

wherein the ratio of the concentration of the above chemical intermediate to that of the N(omega)′-protonated L-arginine at equilibrium state should be greater than 10⁻² (pH<the pKa+2)) for the significant progress of the conversion of the N(omega)′-protonated L-arginine to J-Factor.
2—ΔH°=−26.5 kcal/mol and ΔS°=−0.095 kcal/mol ° K in the bonding of the second proton to the nitrogen of the N(omega)′-protonated guanidino group of N(omega)′-protonated L-arginine. Thus, at 25° C.

Log K₁=(26.5/(1.986×10⁻³×298.15))+((−0.095)/(1.986×10⁻³))=−3.081

pKa=−3.081=>

pKa+2=−1.081 at 25° C.
3—At 20° C.:

Log(K₂/K₁)=(−26.5/1.986×10⁻³)(1/298.15−1/293.15)=0.763=>Log K₂−log K₁=0.763=>Log K₂=0.763−3.081=>pKa=−2.318=>

pKa+2=−0.318
4—pKa+2=0=>pKa=−2=>log K=−2=>T=ΔH°/(ΔS°−R log K)=291.12° Kelvin=>17.97° C.=> a temperature of 17.97° C. is the border of the significant progress of the conversion of said N(omega)′-protonated L-arginine to J-Factor and the temperature at which the pKa is greater than −2 is obtain through the following formula:

T<ΔH/(ΔS°+2R)
5—At 100° C.:

Log(K₂/K₁)=(−26.5/1.986×10⁻³)(1/298.15−1/373.15)=−8.995=>Log K₂−log K₁=−8.995=>Log K₂=−8.995−3.081=>pKa=−12.076=>

pKa+2=−10.076
6—At 75° C.:

Log(K₂/K₁)=(−26.5/1.986×10⁻³)(1/298.15−1/348.15)=−6.427=>Log K₂−log K₁=−6.427=>Log K₂=−6.427−3.081=>pKa=−9.508=>

pKa+2=−7.508
7—At 50° C.:

Log(K₂/K₁)=(−26.5/1.986×10⁻³)(1/298.15−1/323.15)=−3.462=>Log K₂−log K₁=−3.462=>Log K₂=−3.462−3.081=>pKa=−6.543=>

pKa+2=−4.543
8—At 37° C. (physiological temperature):

Log(K₂/K₁)=(−26.5/1.986×10⁻³)(1/298.15−1/310.15)=−1.732=>Log K₂−log K₁=−1.732=>Log K₂=−1.732−3.081=>pKa=−4.813=>

pKa+2=−2.813

Thus, under physiological conditions of human body (pH 7.40 and 37° C.), conversion of N(omega)′-protonated L-arginine to J-Factor is impossible through the above process. (7.40>pKa+2=−2.813)
9—At 30° C.:

Log(K₂/K₁)=(−26.5/1.986×10⁻³)(1/298.15−1/303.15)=−0.738=>Log K₂−log K₁=−0.738=>Log K₂=−0.738−3.081=>pKa=−3.819=>

pKa+2=−1.819
10—At 5° C.:

Log(K₂/K₁)=(−26.5/1.986×10⁻³)(1/298.15−1/278.15)=3.218=>Log K2−log K1=3.218=>Log K2=3.218−3.081=>pKa=0.137=>

pKa+2=2.137
11—At 1° C.:

Log(K₂/K₁)=(−26.5/1.986×10⁻³)(1/298.15−1/274.15)=3.918=>Log K2−log K1=3.918=>Log K₂=3.918−3.081=>pKa=0.837=>

pKa+2=2.837
12—At 0° C.:

Log(K₂/K₁)=(−26.5/1.986×10⁻³)(1/298.15−1/273.15)=4.096=>Log K₂−log K₁=4.096=>Log K2=4.096−3.081=>pKa=1.015=>

pKa+2=3.015
Consequently, in the reaction:

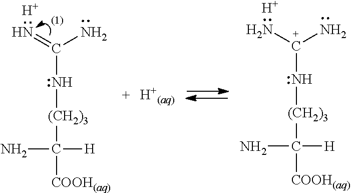

because pH<pKa+2=−1 is impossible in the dilute aqueous solution, said reaction doesn't progress forwardly enough at tempreture> or =30° C., so said temperature prevents the second proton from bonding to the nitrogen of the N(omega)′-protonated guanidino group of N(omega)′-protonated L-arginine and converting N(omega)′-protonated L-arginine to J-Factor in the dilute aqueous solution. In the other hand, a temperature less than 17.97°C. is favorable for the forward progress of the conversion of N(omega)′-protonated L-arginine to J-Factor in the dilute aqueous solution. Because maximum proton(hydronium ion) concentration is necessary for the significant progress of said conversion is 1 mol/lit at temperature less than 17.97° C. and decreases as long as said temperature does.

On the other hand, after the reversible reaction in which the “N(omega)′-protonated L-arginine changes into J-Factor” reaches an equilibrium state at any acidity value or temperature,

$$\frac{J \text{-} Factor concentration}{{N ( omega )}^{′} - protonated L \text{-} arginine concentration} = 0.5 = {K}_{eq}$$

However, it is not required to separate the two bio-molecules because N(omega)′-protonated L-arginine is found much more in the daily diet. While you're administering J-Factor 50 μg per day, N(omega)′-protonated L-arginine is also taken 100 μg per day that is much less than the normal daily dietary intake of the amino acid.

### Example 4

### Another Method of Synthesizing the J-Factor Ex-Vivo

Regarding the pKa=12.48 at 25° C. and what is explained later, N(omega)′-protonated L-arginine is first dissolved in water to obtain a solution. Second, the temperature of the solution is increased or/and the acidity of the solution is decreased to obtain a pH>pKa−2 such as pH>6.30 together with 100° C. at which the guanidino from of L-arginine is enriched enough, and wherein the pKa is a minus logarithm of the acid dissociation constant of the N(omega)′-protonated guanidino group of the N(omega)′-protonated L-arginine in the solution at its temperature, wherein the higher the temperature, the lower is the value of the pKa−2, and wherein a decrease in the acidity is performed through/by adding a base such as barium hydroxide or ammonia to the solution.

Third, all of the derivatives of L-arginine are given time or allowed to reach an equilibrium state at the pH>the pKa−2 according to the following reaction, wherein said time doesn't need to be increased to more than 24 hours:

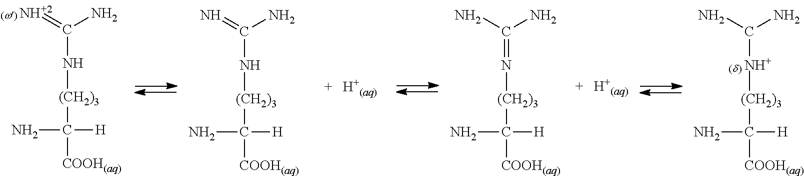

Finally, the acidity of the solution is brought to a pH 7.0 and the temperature of the solution is brought to 25° C., to convert the guanidino group of 1-arginine and the diaminomethyleneamino group of L-2-amino-5-diaminomethyleneamino-pentanoic acid which are in tatoumeric equilibrium and remain after the equilibrium state, into the protonated form, wherein an increase in the acidity is performed by adding an acid such as sulfuric acid or in another way by decreasing the partial pressure of the ammonia.

According the above process, one-third of the initial N(omega)′-protonated L-arginine is converted to J-Factor with a pH 7.0 together with 25° C. as follows:

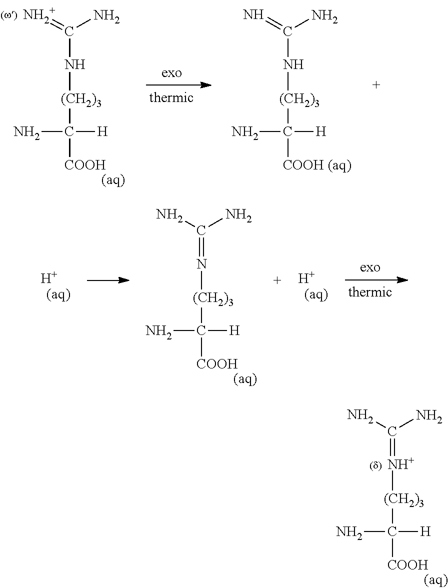

In the above process, the conversion of N(omega)′-protonated L-arginine to Larginine and a proton is endothermic and reversible with ΔH°=51.8 kj/mol=12.3 kcal/mol and ΔS°=16.47 cal/mol ° K. Further, the guanidino form of 1-arginine is more enriched through a more increase in pH. Thus, an increase in temperature or pH increases the chemical intermediate (the guanidino form of 1-arginine) and consequently accelerates production of J-Factor by the process.

However, the ratio of the concentration of the above chemical intermediate (the guanidino form of 1-arginine) to that of the N(omega)′-protonated L-arginine at the equilibrium state should be greater than 10⁻² (pH>pKa−2) for the synthesis of J-Factor through the above process.

Further, in the conversion of N(omega)′-protonated L-arginine to Larginine and a proton, K (equilibrium constant)=Ka (acid dissociation constant). Consequently, the integrated form of the van't Hoff equation is changed as follows:

Log(Ka₂/Ka₁)=(ΔH°/R)(1/T₁−1/T₂)

wherein Ka₁ is the acid dissociation constant at absolute temperature T₁, Ka₂ is the acid dissociation constant at absolute temperature T₂. Now, the calculated ratios of Ka₂ at 100° C., 75° C., 50° C., 37° C. and 30° C. to Ka₁ at 25° C. and their pKa's in the conversion are given below, where pKa is a minus the logarithm of the acid dissociation constant.
1—At 100° C.:

Log(Ka₂/Ka₁)=(12.3/1.986×10⁻³)(1/298.15−1/373.15)=4.18Ka₂=10^{4.18}Ka₁→Ka₂=10^{4.18}×10^{-pKa}^{₁}→Ka₂=10^{4.18}×10^{-12.48}→Ka₂=10^{-8.30}→pKa₂=8.30=>

pKa₂−2=6.30
2—At 75° C.:

Log(Ka₂/Ka₁)=(12.3/1.986×10⁻³)(1/298.15−1/348.15)=2.98Ka₂/Ka₁=10^{2.98}→pKa₂=9.50=>

pKa₂−2=7.50
3—At 50° C.:

Log(Ka₂/Ka₁)=(12.3/1.986×10⁻³)(1/298.15−1/323.15)=1.61Ka₂/Ka₁=10^{1.61} pKa2=10.87=>

pKa₂−2=8.87
4—At 37° C. (human body temperature):

Log(Ka₂/Ka₁)=(12.3/1.986×10⁻³)(1/298.15−1/310.15)=0.80Ka₂/Ka₁=10^{0.80}→pKa₂=11.68=>

pKa₂−2=9.68

Thus, under physiological conditions (pH 7.40 and 37° C.), conversion of N(omega)′-protonated L-arginine to J-Factor is impossible through the above process. (7.40<pKa₂−2=9.68)
5—At 30° C.:

Log(Ka₂/Ka₁)=(12.3/1.986×10⁻³)(1/298.15−1/303.15)=0.34Ka₂/Ka₁=10^{0.34}→pKa₂=12.14=>

pKa₂−2=10.14
6—At 25° C.:

pKa₁=12.48=>pKα₁−2=10.48

Therefore, except in the first case, a conversion of N(omega)′-protonated L-arginine to J-Factor at pH 7.00 is practically impossible through the above process at the other temperatures mentioned above. (7.00<pKa−2).

Hence a composition having a N(omega)′-protonated guanidino (diaminomethylideneamino) group without resonance (mesomerism) is converted to its derivative having a N(delta)-protonated diaminomethyleneamino group without resonance (mesomerism) through all of the methods mentioned above as follows provided that the composiom has molecular mass more than or equal to 174.2:

### Similar to the Example 4:

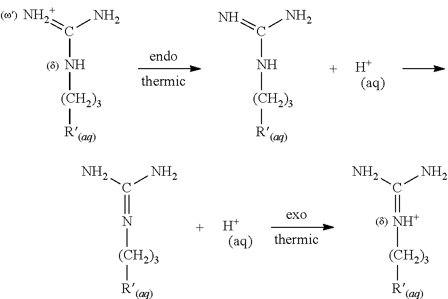

In this reaction, initially the temperature of the solution is increased or/and the acidity of the solution is decreased as shown in the example 4,

### Similar to the Example 3:

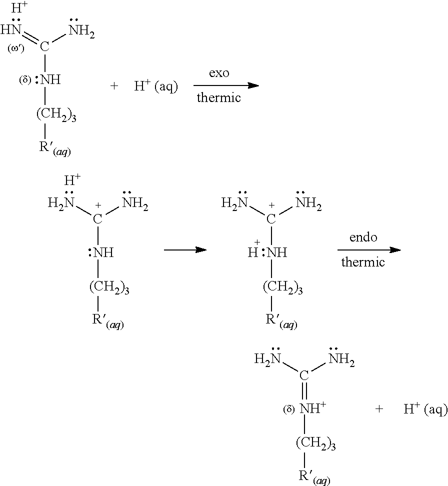

In the above reaction, a first kelvin temperature of the solution is decreased or brought to a second kelvin temperature less than ΔH°/(ΔS°+2R) to obtain a pKa>−2 and then an acidity of the solution is increased to obtain a pH<the pKa+2 as shown the example 3.

Both of the above reactions are reversible at any stage, wherein R′ is a chemical group bonding to —(CH₂)₃— through a single C—C bond, wherein the R′ contains a carboxyl group or its ionized form, wherein the R′ contains an amino group or its ionized form. in addition, in both of the above reactions, the first and second acid dissociation constants of the diprotonated form of said guanidino group (diaminomethylideneamino) are similar or equal to those of L-arginine, due to the absence of resonance effect in the substituent “—(CH₂)₃—R′” and brief change in its inductive effect in the spite of the “R′” change under the above limitations. Thus, with regard to Van't Hoff equation practically in an aqueous solution:
1—at the first acid dissociation of said diprotonated form→ΔH°=26.5 kcal/mol, ΔS°=95 cal/mol ° K=>pKa=−3.08 at 25° C.
2—at its second acid dissociation→ΔH°=51.8 kj/mol=12.3 kcal/mol, ΔS°=16.47 cal/mol ° K=>pKa=12.48 at 25° C.

Thus, the peptide having a N(omega)′-protonated guanidino group without resonance is converted into the peptide having a N(delta)-protonateddiaminomethyleneamino group without resonance through all of the methods mentioned above. Furthermore, the conversion of N(omega)′-protonated L-arginine to J-Factor happens only through the chemical intermediate and its rate depends on the intermediate concentration. When the chemical intermediate is insignificant as observed under physiological conditions (pH 7.40 and 37° C.), the conversion is too slow to be possible. In addition, neither of the guanidino and diaminomethyleneamino groups as monoprotonated have the tautomeric quality unlike their non-protonated form. Anyway, the interconversion of the tautomers is rapid. In fact, the interconversion of the N(omega)′-protonated guanidino and N(delta)-protonated diaminomethyleneamino groups without mesomerism is impossible, unless they are initially converted as depotonated or diprotonated.

In all of the methods of this application, the pH is measured by the pH meter or indicator. In addition, any composition administrated orally should have a pH not damaging to alimentary canal after eating like 7.0 which is the safest pH and that injected should have a pH about 7.0. Anyway, physiological pH which is 7.40 is the safest pH for injection. In addition, both acidic (pH<4) and alkaline (pH>10) solutions are capable of inducing a chemical burn. Thus, with regard to the confidence border, synthetic J-Factor and its analogues in the embodiments herein were formulated at pH 5-9 not producing damage to alimentary canal after eating like 7.0 for oral administration, and at pH about 7.0 like physiological pH for injection. Otherwise, it can't administered as a therapeutic composition.

In fact, a pH less than 2 or more than 11.5 is harmful for alimentary canal after oral administration. Acids with a pH lower than 2 cause coagulate necrosis and alkali agents with a pH higher than 11.5 cause liquefactive necrosis, allowing deeper penetration of the chemical. The materials which remain from all of the synthesis methods beside J-Factor and its analogues formulated as a therapeutic composition must not be at a toxic level and damage a living body. Therefore, they should be separable from J-Factor and its analogues to reach a non-toxic level, in other words, chemicals and solvents that are toxic or produce toxic materials are not used in synthesis of J-Factor, its analogues or any other therapeutic compositions unless the method to remove them to reach a non-toxic level is considered.

In addition, synthetic J-Factor and its analogues should be recoverable from the solvent to be concentrated enough and used as a therapeutic composition. Therefore, a solvent can't used in synthesis of J-Factor and its analogues without considering said fact.

In the present application, except in the Example 5 and 6, solvent is only water which is non-toxic and separable from f-Factor and its analogues through vaporizing by heat or a decrease in pressure.

In all of the synthesis methods of this application, the composition selected as a reactant, is dissolvable in the reaction solvent. Otherwise, the reaction can't progress forwardly.

### Example 5

1—The first total method of converting a N(omega)′-protonated guanidino(diaminomethylideneamino) group without resonance(mesomerism) of a composition having molecular mass more than or equal 174.2 u to a N(delta)-protonated diaminomethyleneamino group without resonance(mesomerism) in a liquid solvent dissolving the composition like water, concentrated sulfuric acid, super acids and so on, comprises steps of dissolving the composition in the solvent to obtain a solution, then adjusting or bringing the solution to an arbitrary temperature, controlling or bringing the acidity of the solution to a Hammett acidity function (H₀) or to a pH which is less than pKa+2 at the arbitrary temperature, wherein the pKa is a minus logarithm of the first acid dissociation constant of the diprotonted form of the guanidino group in an aqueous solution at the arbitrary temperature, wherein the lower the arbitrary temperature, the higher is the value of the pKa+2, and wherein an increase in the acidity is performed through/by adding an acid such as sulfuric acid or hydrochloric acid to the solution. Then the derivations of the N(omega)′-protonated guanidino group is given time to reach an equilibrium state at the Hammett acidity function (H₀) or pH less than the pKa+2 together with the arbitrary temperature, wherein said time doesn't need to be increased to more than 24 hours. Finally, the acidity of the solution is adjusted to a pH value of 7.0 and the arbitrary temperature of the solution is adjusted to 25° C. to convert the diprotonated forms of the guanidino group which remain after the equilibrium state to a monoprotonated form and wherein a decrease in the acidity is performed through or by adding a base such as barium hydroxide to the solution or in another way by using anion exchange resin column chromatography, wherein the composition has a formula represented by

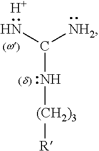

wherein R′ is a chemical group bonding to —(CH2)₃— through a single C—C bond, and wherein the R′ contains a carboxyl group or its ionized form, wherein the R′ contains an amino group or its ionized form.
2—According the above process, one-third of the initial N(omega)′-protonated guanidino group is converted to the N(delta)-protonated diaminomethyleneamino group with a pH 7.0 together with 25° C.
3—With regard to the example 5, since the solvent system containing 60 wt. % H₂SO₄ in water has H₀ value −4.32, N(omega)′-protonated L-arginine is converted to J-Factor in the solvent system at a temperature less than 48.26° C. with finally bringing the acidity to a pH value of 7.0 together with 25° C., based on the following calculation:

pKa+2=H₀=>pKa=H₀−2=>logK=H₀−2=>T=ΔH°/(ΔS°−R Log K)=ΔH′)/(ΔS°−R(H₀−2))=>T=−26.5/(−0.095−(1.986×10⁻³)(−4.32−2))=>T=321.41=>48.26° C.

Therefore, to convert N(omega)′-protonated L-arginine to J-Factor based on the Example 5, first dissolve N(omega)′-protonated L-arginine in the liquid solvent system containing a strong acid such as sulfuric acid, hydrochloric acid and so on with a known negative Hammett acidity function “H₀” to obtain a solution. Secondly, the Kelvin temperature of the solution is adjusted or brought to T<ΔH°/(ΔS°−R(H₀−2)). Third, the derivations of the N(omega)′-protonated L-arginine are given time to reach an equilibrium state at the T<ΔH°/(ΔS*−R(H₀−2)), wherein said time doesn't need to be increased to more than 24 hours, and wherein the lower the H₀, the higher is the maximum of the T, wherein the ΔH° is a change in enthalpy and the ΔS° is a change in entropy in a standard temperature and pressure conditions and wherein the standard temperature is 25° C. and the standard pressure is 1 atmospheric pressure, when a N(omega)′-protonated guanidino group of the N(omega)′-protonated L-arginine is converted to diprotonated form of the guanidino group in an aqueous solution. Then the acidity of the solution is finally adjusted to be or brought to a pH value of 7.0 at 25° C. The above process is true and holds good for the synthesis of all of the J-Factor analogues as mentioned in the number 40.
4—However, the solvent system containing a strong acid with a known Hammett acidity function “H₀” for a lot of molar concentrations is mentioned/given in the tables 1 and 2, and wherein a unknown H₀ value for a molar concentration is calculated through a linear interpolation technique. If the two known points are given by the coordinates (x₀, y₀) and (x₁, y₁), the linear interpolant is the straight line between these points, wherein x₀ and x₁ are two subsequent molar concentrations with known H₀ values of y₀ and y₁, respectively. For a value x in the interval (x₀, x₁), the value y along the straight line is given from the equation

$$\frac{y - {y}_{0}}{x - {x}_{0}} = \frac{{y}_{1} - {y}_{0}}{{x}_{1} - {x}_{0}}$$

Solving this equation for y, which is the unknown value at x, gives

$$y = {y}_{0} + ( {y}_{1} - {y}_{0} ) \frac{x - {x}_{0}}{{x}_{1} - {x}_{0}}$$

wherein the value x is a molar concentration and the value y is the value at the value x.
5—With respect to the following tables, the line between the two consequent molar concentration is slightly curved. Thus, the H₀ value calculated through the equation is insignificantly different from the true value.

TABLE 1
The H₀ values for the system of sulfuric acid and water

| Concentration of sulfuric | |
|---|---|
| acid (mol/lit) at 25° C. | H₀ |
| 0.1 | 0.83 |
| 0.50 | 0.13 |
| 1.0 | −0.26 |
| 1.5 | −0.56 |
| 2.0 | −0.84 |
| 2.5 | −1.12 |
| 3.0 | −1.38 |
| 3.5 | −1.62 |
| 4.0 | −1.85 |
| 4.5 | −2.06 |
| 5.0 | −2.28 |
| 5.5 | −2.51 |
| 6.0 | −2.76 |
| 6.5 | −3.03 |
| 7.0 | −3.32 |
| 7.5 | −3.60 |
| 8.0 | −3.87 |
| 8.5 | −4.14 |
| 9.0 | −4.40 |
| 9.5 | −4.65 |
| 10.0 | −4.89 |
| 10.5 | −5.15 |
| 11.0 | −5.41 |
| 11.5 | −5.67 |
| 12.0 | −5.93 |
| 12.5 | −6.18 |
| 13.0 | −6.44 |
| 13.5 | −6.70 |
| 14.0 | −6.96 |
| 14.5 | −7.22 |
| 15.0 | −7.47 |
| 15.5 | −7.72 |
| 16.0 | −7.98 |
| 16.5 | −8.23 |
| 17.0 | −8.49 |
| 17.5 | −8.75 |
| 18.0_{(96vol %)} | −9.04 |
| 18.1 | −9.13 |
| 18.2 | −9.22 |
| 18.3 | −9.36 |
| 18.4 | −9.56 |
| 18.5 | −9.89 |
| 18.61 | −11.10 |
| 18.75(pure) | −11.93 |

TABLE 2
The H₀ values for the system of hydrochloric acid and water

| Concentration of hydrochloric | |
|---|---|
| acid (mol/lit) at 25° C. | H₀ |
| 0.1020 ≈ 0.1 | 1.06 |
| 0.396 ≈ 0.4 | 0.36 |
| 0.792 ≈ 0.8 | 0.02 |
| 1.188 ≈ 1.2 | −0.19 |
| 1.584 ≈ 1.6 | −0.36 |
| 1.980 ≈ 2.0 | −0.53 |
| 2.376 ≈ 2.4 | −0.68 |
| 3.168 ≈ 3.2 | −0.98 |
| 3.96 ≈ 4.0 | −1.28 |
| 4.75 ≈ 4.8 | −1.57 |
| 5.94 ≈ 5.9 | −1.98 |
| 7.13 ≈ 7.0 | −2.47 |
| 7.92 ≈ 7.9 | −2.84 |

### Example 6

1—Now, a summary about the function H_:

The function of H- is similar to H₀ for strong bases and extends the measure of Brønsted-Lowry acidity beyond a pH value of 14. The function H_ is ability of the solution to remove a proton of a reactant like N(omega)′-protonated L-arginine. The value H_is calculated with the following equation:

H_=pKa+log([B⁻]/[BH])

Where BH is a weak acid used as an acid-base indicator, and B⁻ is its conjugate base, where pKa is the negative logarithm of the acid dissociation constant of BH in water.

In dilute aqueous solution(pH 0-14), the predominant acid species is the hydrated hydrogen ion (H₃O⁺). In this case, H₀ and H_ are equivalent to pH values determined by Henderson-Hasselbalch equation.

2—The second total method of converting a N(omega)′-protonated guanidino group without resonance of a composition having molecular mass more than or equal to 174.2 u to a N(delta)-protonated diaminomethyleneamino group without resonance in a liquid solvent dissolving the composition, comprises steps of dissolving the composition in the liquid solvent to obtain a solution, then bringing or adjusting a temperature of the solution to an arbitrary temperature, decreasing the acidity of the solution to a H_or pH which is more than pKa−2 at the arbitrary temperature, wherein the pKa is a minus logarithm of the acid dissociation constant of the N(omega)′-protonated guanidino group of the composition in an aqueous solution at the arbitrary temperature. Then the derivations of the N(omega)′-protonated guanidino group are given time or allowed to reach an equilibrium state at the H_ or pH which is more than pKa−2 together with the arbitrary temperature, wherein said time doesn't need to be increased to more than 24 hours. Then the acidity of the solution is brought to a pH value of 7.0 and the arbitrary temperature of the solution to 25° C., and wherein the composition has a formula represented by

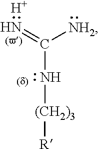

wherein R′ is a chemical group bonding to —(CH₂)₃— through a single C—C bond, wherein the R′ contains a carboxyl group or its ionized form, wherein the R′ contains an amino group or its ionized form.

In this application
1—an increase in the acidity is performed through adding an acid such as sulfuric acid or hydrochloric acid to the solution or in another way by decreasing the partial pressure of a gaseous base like ammonia
2—a decrease in the acidity is performed by adding a base such as barium hydroxide or ammonia to the solution or in another way by using anion exchange resin column chromatography
3—all of the synthesis methods of J-Factor and its analogues are reversible at any chemical stage.

If the J-Factor of the embodiments herein was synthesized from N(omega)′-protonated L-arginine in another route in the body other than from GH and IGF-1 axis, the autocrine-paracrine IGF-1 wouldn't decreased along with aging.

The J-factor in the embodiments herein is formulated into a therapeutic composition in a suitable pharmaceutical acceptable for administering to patients suffering from age-related disorders and diseases. The J-factor of the embodiments herein is prepared for oral administration by mixing J-factor having the desired degree of purity with physiologically acceptable carriers. Such carriers will be nontoxic to recipients at the dosages and concentrations employed. These therapeutic compositions administered to the patients selected from the group consisting of bone-fracture, wound healing, type-II diabetic, neurodegenerative conditions, cancer, aging, and muscle wasting diseases.

According to an embodiment herein, the therapeutic composition for administration is prepared by mixing a required concentration of J-factor and L-agrinine having a desired purity with suitable pharmaceutical acceptable carriers. These therapeutic compositions can further include protein, antioxidants and other such agents which are beneficial and useful to reduce the signs of the aging and age-related disorders.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A therapeutic composition of N(delta)-protonated L-2-amino-5-diaminomethyleneamino-pentanoic acid formulated at pH 5-9 having a structural formula represented by 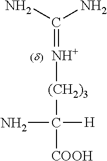
where N is Nitrogen, O is Oxygen, H is Hydrogen, C is Carbon, ═ is a double bond, — is a single bond, —NH₂ represents an amino group, —CH2- represents Methylene, —COOH is a Carboxyl group, and wherein said therapeutic composition includes ionized forms of said N(delta)-protonated L-2-amino-5-diaminomethyleneamino-pentanoic acid in a carboxyl group or in a 2-amino group, and wherein said ionized forms are in equilibrium with said N(delta)-protonated L-2-amino-5-diaminomethyleneamino-pentanoic acid, and wherein a chemical structure of said therapeutic composition has a N(delta)-protonated L-2-amino-5-diaminomethyleneamino group, wherein said therapeutic composition has a second structural formula, and wherein the second structural formula is represented by

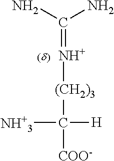

2. The therapeutic composition of claim 1, wherein said therapeutic composition is derived from N(omega)′-protonated 1-arginine having a third structural formula and wherein the third structural formula is represented by 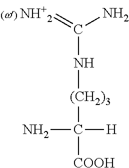
and wherein said therapeutic composition is derived from N(omega)′-protonated 1-arginine by bringing a first Kelvin temperature of an aqueous solution of said N(omega)′-protonated 1-arginine to a second Kelvin temperature and increasing the acidity of said aqueous solution to obtain a preset value of pH and wherein the second Kelvin temperature is less than ΔH°/(ΔS°+2R), and wherein said ΔH° is a change in an enthalpy and wherein said ΔS° is a change in an entropy under a standard temperature and pressure conditions and wherein the standard temperature and pressure conditions includes a temperature of 25° C. and a pressure of 1 atmospheric pressure, and wherein said R is gas constant and wherein the preset value of pH is less than a sum of pKa+2, and wherein said pKa is a minus logarithm of a first acid dissociation constant of a diprotonated guanidino group of an intermediate chemical compound having a fourth structural formula in said aqueous solution at the second Kelvin temperature and wherein the fourth structural formula is represented by 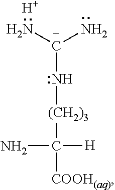
wherein said fourth structural formula includes ionized forms in a carboxyl group or 2-amino group which are in equilibrium with said fourth structural formula, and giving time to derivatives of said N(omega)′-protonated 1-arginine to reach an equilibrium state in said aqueous solution at said pH which is less than the sum of pKa+2 at said second Kelvin temperature, and wherein said acidity of said aqueous solution is brought to a pH value of 7.0 and the temperature of said aqueous solution to 25° C. after reaching said equilibrium state, and wherein said N(omega)′-protonated 1-arginine includes ionized forms in a carboxyl or 2-amino group which are in equilibrium with said N(omega)′-protonated 1-arginine, and wherein said N(omega)′-protonated 1-arginine has a N(omega)′-protonated guanidino group, and wherein said therapeutic composition stimulates a synthesis and secretion of autocrine-paracrine IGF-1 in tissues in a living body.

3. The therapeutic composition of claim 2, wherein said therapeutic composition is produced by dislocating a double bond in said N(omega)′-protonated guanidino group of said N(omega)′-protonated L-arginine which is represented by a following reaction: 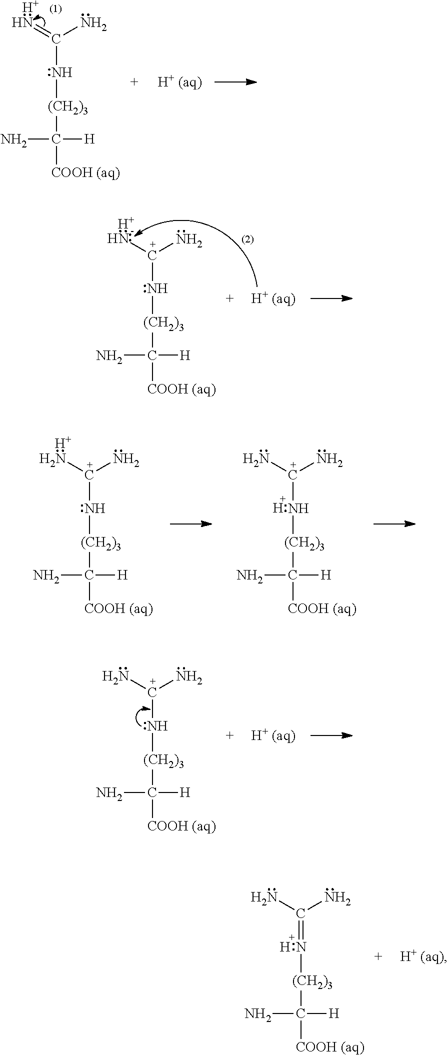
wherein said reaction is reversible at any stage.

4. The therapeutic composition of claim 1, wherein said N(delta)-protonated diaminomethyleneamino group increases an expression of IGF-1mRNA in all cells of a living body.

5. The therapeutic composition of claim 1, wherein said therapeutic composition is ex-vivo synthesized from N(omega)′-protonated L-arginine by increasing a temperature of an aqueous solution containing said N(omega)′-protonated L-arginine and/or decreasing an acidity of said aqueous solution containing said N(omega)′-protonated L-arginine to obtain a pH value which is greater than a value of pKa−2 and wherein said pKa is a minus logarithm of an acid dissociation constant of the N(omega)′-protonated guanidino group of said N(omega)′-protonated L-arginine at said temperature of said aqueous solution, allowing derivatives of said N(omega)′-protonated L-arginine to reach an equilibrium state in said aqueous solution at said pH which is greater than said value of pKa−2, and bringing the acidity of the aqueous solution to a pH value of 7.0 and the temperature of the aqueous solution to 25° C. and wherein said N(omega)′-protonated 1-arginine includes ionized forms, which are in equilibrium with said N(omega)′-protonated 1-arginine.

6. The therapeutic composition of claim 1, wherein said therapeutic composition is ex-vivo synthesized from a reactant by bringing a temperature and an acidity of an aqueous solution with a pH which is greater than a value of pKa−2 sequentially to 25° C. and a pH value of 7.0, wherein said pKa is a minus logarithm of an acid dissociation constant of a N(omega)′-protonated guanidino group of N(omega)′-protonated L-arginine at said temperature of said aqueous solution, wherein said aqueous solution contains said reactant, and wherein said reactant is a sum of L-2-amino-5-diaminomethyleneamino-pentanoic acid and ionized forms of said L-2-amino-5-diaminomethyleneamino-pentanoic acid in a carboxyl group or 2-amino group which are in equilibrium with said L-2-amino-5-diaminomethyleneamino-pentanoic acid.

7. The therapeutic composition of claim 2, wherein said therapeutic composition improves hair growth and muscular hypertrophy and restores the muscle mass, increases bone density, decreases fatty tissue, improves eye's central vision, decreases cellular proptosis, and improves skin elasticity

8. The therapeutic composition of claim 1, wherein said therapeutic composition is synthesized from N(omega)′-protonated L-arginine through a process comprising the steps of:
dissolving said N(omega)′-protonated L-arginine in a solvent system containing a strong acid with a known Hammett acidity function “H₀” to obtain a solution;
bringing a first Kelvin temperature of said solution to a second Kelvin temperature, and wherein the second Kelvin temperature is less than ΔH°/(ΔS°−R(H₀−2)), and wherein said R is gas constant, and wherein said ΔH° is a change in enthalpy and wherein said ΔS° is a change in entropy change under a standard temperature and pressure conditions and wherein the standard temperature and pressure conditions includes a temperature of 25° C. and a pressure of 1 atmospheric pressure, when a N(omega)′-protonated guanidino group of said N(omega)′-protonated L-arginine is converted to a diprotonated form of said guanidino group in water, allowing derivatives of said N(omega)′-protonated L-arginine to reach an equilibrium state in said solution at said second Kelvin temperature by providing time, and bringing the acidity of said solution to a pH value of 7.0 and the temperature of said solution to 25° C., wherein said N(omega)′-protonated 1-arginine includes ionized forms in a carboxyl or 2-amino group, which are in equilibrium with said N(omega)′-protonated 1-arginine.

9. The therapeutic composition of claim 2, wherein said therapeutic composition stimulates a synthesis and a secretion of IGF-1

10. A therapeutic compound having a formula 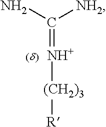
wherein said R′ is a chemical group bonding to said —(CH2)₃-through a single C—C bond, and wherein said R′ contains a carboxyl group or an ionized form of said carboxyl group, and wherein said R′ contains an amino group or an ionized form of said amino group, and wherein said therapeutic compound has a molecular mass of more than or equal to 174.2 u.

11. The therapeutic compound of claim 10, wherein said therapeutic compound is derived from a composition having a N(omega)′-protonated guanidino group through a method comprising the steps of:
dissolving said composition in a solvent system containing a strong acid with a known Hammett acidity function “H₀” to obtain a solution; bringing a first Kelvin temperature of said solution to a second Kelvin temperature, and wherein the second Kelvin temperature is less than ΔH°/(ΔS°−R(H₀−2)), and wherein said R is gas constant, and wherein said ΔH° is a change in enthalpy and wherein said ΔS° is a change in entropy change under a standard temperature and pressure conditions and wherein the standard temperature and pressure conditions includes a temperature of 25° C. and a pressure of 1 atmospheric pressure, when said N(omega)′-protonated guanidino group of said composition is converted to a diprotonated form of said guanidino group in water, allowing derivatives of said N(omega)′-protonated guanidino group to reach an equilibrium state in said solution at said second Kelvin temperature by providing time, and bringing the acidity of said solution to a pH value of 7.0 and the temperature of said solution to 25° C., and wherein said composition has a chemical structure represented by

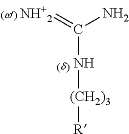

12. The therapeutic compound of claim 10, wherein said therapeutic compound is derived from a composition having a N(omega)′-protonated guanidino group through a method comprising the steps of:
increasing a temperature of an aqueous solution containing said composition having said N(omega)′-protonated guanidino group and/or decreasing an acidity of said aqueous solution containing said composition having said N(omega)′-protonated guanidino group to obtain a pH which is more than a value of pKa−2, and wherein said pKa is a minus logarithm of an acid dissociation constant of said N(omega)′-protonated guanidino group at said temperature of said aqueous solution, giving time to derivatives of said N(omega)′-protonated guanidino group to reach an equilibrium state in said aqueous solution at said pH which is more than said value of pKa−2; and bringing said temperature of said aqueous solution to 25° C. and the acidity of said aqueous solution to a pH value of 7.0, wherein said composition has a structural formula

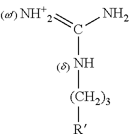

13. The therapeutic compound of claim 10, wherein said therapeutic compound stimulates a synthesis and a secretion of IGF-1

14. A method of converting a composition having a N(omega)′-protonated guanidino group to a product having a N(delta)-protonated diaminomethyleneamino group, the method comprising the steps of:
bringing a first Kelvin temperature of an aqueous solution containing said composition having said N(omega)′-protonated guanidino group to a second Kelvin temperature, and wherein the second Kelvin temperature is less than ΔH°/(ΔS°+2R), and wherein said ΔH° is a change in enthalpy and wherein said ΔS° is a change in entropy under a standard temperature and pressure conditions and wherein the standard temperature and pressure conditions include a temperature of 25° C. and a pressure of 1 atmospheric pressure, when said N(omega)′-protonated guanidino group is converted to a diprotonated form of said guanidino group in said aqueous solution, and wherein said R is gas constant;
increasing an acidity of said aqueous solution containing said composition having said N(omega)′-protonated guanidino group to obtain a pH which is less than a value of pKa+2, and wherein said pKa is a minus logarithm of a first acid dissociation constant of a diprotonated form of said guanidino group of said composition in said aqueous solution at said second Kelvin temperature, allowing derivatives of said N(omega)′-protonated guanidino group to reach an equilibrium state in said aqueous solution at said pH which is less than said value of pKa+2 together and at said second Kelvin temperature by providing time, and bringing said acidity of said aqueous solution to a pH value of 7.0 and the temperature of said aqueous solution to 25° C., and wherein said composition has a formula represented by 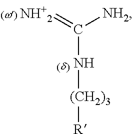
wherein said R′ is a chemical group bonding to said —(CH2)₃-through a single C—C bond, and wherein said R′ contains a carboxyl group or an ionized form of said carboxyl group, and wherein said R′ contains an amino group or an ionized form of said amino group, and wherein said composition has a molecular mass of more than or equal to 174.2 u.

15. The method according to claim 14, wherein said N(delta)-protonated diaminomethyleneamino group stimulates a synthesis of IGF-1mRNA and IGF-1 in the cell.

16. The method according to claim 14, wherein said product is a therapeutic composition.
